(19) United States
(12) Patent Application Publication
(10) Pub. No.: US 2006/0188566 A1
(43) Pub. Date: Aug. 24, 2006

(54) Nanoparticulate formulations of docetaxel and analogues thereof

(75) Inventors: Gary Liversidge, Westchester, PA (US); Scott Jenkins, Downingtown, PA (US); Elaine Liversidge, Westchester, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/361,055

(22) Filed: Feb. 24, 2006

Related U.S. Application Data
(60) Provisional application No. 60/655,934, filed on Feb. 24, 2005.

(51) Int. Cl. A61K 31/337; A61K 9/48; A61K 9/20

(52) U.S. Cl. 424/451; 549/510; 514/449; 977/907; 977/906; 424/464

(57) ABSTRACT

Described are nanoparticulate docetaxel or analogue thereof compositions. The compositions, which comprise a nanoparticulate docetaxel or analogue thereof and at least one surface stabilizer, can be used in the treatment of cancer.

### FIELD OF THE INVENTION

The present invention is directed to nanoparticulate compositions of docetaxel and analogues thereof, methods of making such compositions, and the use of such nanoparticulate compositions in the treatment of cancer, and in particular, breast, ovarian, prostate, and lung cancer.

### BACKGROUND OF THE INVENTION

A. Background Regarding Docetaxel and Analogues Thereof

Taxoids or taxanes are compounds that inhibit cell growth by stopping cell division, and include docetaxel and paclitaxel. They are also called antimitotic or antimicrotubule agents or mitotic inhibitors.

Taxoid-based compositions having anti-tumor and anti-leukemia activity, and the use thereof, are described in U.S. Pat. No. 5,438,072. U.S. Pat. No. 6,624,317 refers to the preparation of taxoid conjugates for use in the treatment of cancer. FIG. 1A of U.S. Pat. No. 5,508,447 to Magnus (the “Magnus patent”) shows the structure and numbering of the taxane ring system. The Magnus patent is directed to the synthesis of taxol for use in cancer treatment. U.S. Pat. Nos. 5,698,582 and 5,714,512 relate to taxane derivatives used in pharmaceutical compositions suitable for injection as anti-tumor and anti-leukemia treatments. U.S. Pat. Nos. 6,028,206 and 5,614,645 relate to the preparation of taxol analogues that are useful in the treatment of cancer. U.S. Pat. Nos. 4,814,470 and 5,411,984 both relate to the preparation of certain taxol derivatives for use in the treatment of cancer.

Nanoparticulate compositions of paclitaxel are described in U.S. Patent Nos. U.S. Pat. Nos 5,494,683 and 5,399,363. These patents do not describe nanoparticulate docetaxel formulations.

The chemical structure of paclitaxel is shown below:

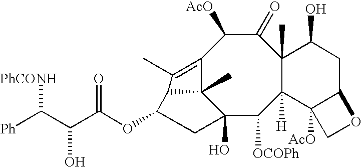

Docetaxel is a semi-synthetic, antineoplastic agent belonging to the taxoid family. Docetaxel is a white to almost-white powder with an empirical formula of C₄₃H₅₃NO₁₄·3H₂O, and a molecular weight of 861.9. It is highly lipophilic and practically insoluble in water. The chemical name for docetaxel is (2R, 3S)-N-carboxy-3-phenylisoserine, N-tert-butyl ester, 13-ester with 5β-20-epoxy-1, 2α, 4, 7β, 10β, 13α-hexahydroxytax-11-en-9-one 4-acetate 2- benzoate, trihydrate. Docetaxel is prepared by semisynthesis beginning with a precursor (taxoid 10-deacetylbaccatin III) extracted from the renewable needle biomass of yew plants. The structure of docetaxel, which is shown below, differs significantly from that of paclitaxel:

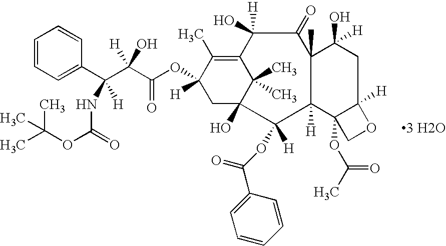

The unique chemical structure of docetaxel contains 2 modifications relative to paclitaxel: (1) A hydroxy group replaces an acetyl group at C-10 on the taxol B ring; and (2) C-13 side chain variations (e.g., an N-tert-butoxycarbonyl group instead of the N-benzoyl group on the taxol side chain). These significant structural differences results in paclitaxel and docetaxel having different activities. For example, docetaxel is more potent than paclitaxel. Angelo et al., “Docetaxel versus paclitaxel for antiangiogenesis,” J. Hematother. Stem. Cell Res., 11(1): 103-18 (2002). In addition, in a study comparing the induction of COX-2 expression by paclitaxel and docetaxel, it was found that in contrast to the similar kinetic and concentration-response profiles for paclitaxel-induced COX-2 expression in human and murine cells, docetaxel induces COX-2 expression only in human monocytes, and not in murine cells. Cassidy et al., Clin. Can. Res., 8:846-855 (2002).

Moreover, the mechanism of action of docetaxel differs from that of paclitaxel. Docetaxel disrupts the microtubular network in cells that is essential for mitosis to occur as well as effecting the normal microtubule-regulated cellular activities. This mechanism of action results in less severe side effects than paclitaxel.

Docetaxel is marketed as TAXOTERE® Injection Concentrate by Aventis Pharmaceuticals (Bridgewater, N.J.). TAXOTERE® is sterile, non-pyrogenic, and is available in single-dose vials containing 20 mg (0.5 mL) or 80 mg (2.0 mL) docetaxel (anhydrous). Each mL contains 40 mg docetaxel (anhydrous) and 1040 mg polysorbate 80. TAXOTERE® Injection Concentrate requires dilution prior to use. A sterile, non-pyrogenic, single-dose diluent is supplied for that purpose. The diluent for TAXOTERE® contains 13% ethanol in Water for Injection, and is supplied in vials.

The presence of polysorbate 80 and ethanol, which are used to increase the solubility of docetaxel, can cause adverse effects. Because of the adverse hypersensitivity associated with TAXOTERE®, premedication with oral dexamethasone for three days beginning 24 hours prior to chemotherapy is advised. Polysorbate 80 has been implicated in severe hypersensitivity reactions characterized by hypotension and/or bronchospasm or generalized rash/erythema, which occurred in 2.2% (2/92) of patients who received the recommended 3-day dexamethasone premedication. In addition, docetaxel injection requires dilution prior to use. A sterile, non-pyrogenic single-dose diluent must be supplied for that purpose. As noted above, the diluent for TAXOTERE® injectable formulations contains 13% ethanol in water for injection, which must be supplied along with the drug.

Docetaxel can cause a decrease in the number of blood cells in a patient's bone marrow, and the drug also can cause liver damage. In addition, cases of hypersensitivity have been observed with TAXOTERE® administration. Symptoms include hypotension and/or bronchospasm, and generalized rash/erthema. Some over dosage cases have also been observed (dosages of 150-200 mg/m²). Some complications associated with this include bone marrow suppression, peripheral neurotoxicity, and mucositis.

The solvents polysorbate 80 and ethanol are responsible at least in part for the hypersensitivity reactions seen with TAXOTERE® administration. Administration of steroids and other histamine-blocking drugs as premedications has reduced the incidence and severity of these reactions, but the adverse events related to the premedications (e.g., Cushing's syndrome, infectious complications, hyperglycemia, hypertension, and psychiatric effects including steroid-induced psychoses) are also of concern, especially with chronic administration. The solvents also contribute to the leaching of plasticizers from polyvinyl chloride (PVC) bags and tubing and possibly other adverse effects experienced with these agents (e.g., neuropathy and tumor cell resistance).

One alternative drug formulation having higher water solubility utilized with paclitaxel is albumin bound paclitaxel (ABRAXANE®). However, this drug formulation requires covalently binding paclitaxel to albumin, which can therefore alter the properties of paclitaxel. For example, in phase I and II clinical trials with albumin-bound paclitaxel, solvent-mediated toxicities were not seen, premedications were not required, and the drug was infused over only 30 minutes. However, the pharmacokinetic profile of this agent appeared to be linear in the phase I trial, differing from traditional paclitaxel, which exhibits nonlinear pharmacokinetics “Abraxane (paclitaxel protein-bound particles for injectable suspension [albumin-bound]) product information,” Abraxis Oncology (Schamburg, Ill.), January 2005.

In clinical pharmacological terms, docetaxel is an antineoplastic agent that acts by disrupting the microtubular network in cells that is essential for mitotic and interphase cellular functions. Docetaxel binds to free tubulin and promotes the assembly of tubulin into stable microtubules while simultaneously inhibiting their disassembly. This leads to the production of microtubule bundles without normal function and to the stabilization of microtubules, which results in the inhibition of mitosis in cells. Docetaxel's binding to microtubules does not alter the number of protofilaments in the bound microtubules, a feature which differs from most spindle poisons currently in clinical use. Physicians' Desk Reference, 58^{th} Ed., pp. 3, 307, 771-78 (Thompson PDR, Montvale, N.J., 2004).

TAXOTERE® (docetaxel) was first approved in 1996 by the U.S. Food and Drug Administration for use in locally advanced or metastatic breast cancer after failure of prior anthracycline chemotherapy. The drug was then approved in 1999 for second-line use in locally advanced or metastatic non-small cell lung cancer (NSCLC). On November 2002, the U.S. Food and Drug Administration approved TAXOTERE® (docetaxel) for use in combination with cisplatin for the treatment of patients with unresectable, locally advanced or metastatic non-small cell lung cancer (NSCLC) who have not previously received chemotherapy for this condition. In 2004, TAXOTERE®, in combination with prednisone, was approved for the treatment of patients with androgen-independent (hormone-refractory) metastatic prostate cancer. In addition, TAXOTERE®, in combination with doxorubicin and cyclophosphamide, has been approved by the U.S. FDA for the adjuvant treatment of patients with operable, node-positive breast cancer. TAXOTERE® continues to be tested in clinical trials for various stages of many types of cancer.

In phase I studies, the pharmacokinetics of docetaxel (TAXOTERE®) were evaluated in cancer patients after administration of doses ranging from 20 mg/m to 115 mg/m². Following intravenous doses of 70 mg/m² to 115 mg/m², the pharmacokinetics of docetaxel were dose-independent and consistent with a 3-compartment model, with mean population α, β, γ half-lives of 4 minutes, 36 minutes, and 11.1 hours, respectively. The approved dosing range for TAXOTERE® is 60 mg/m² to 100 mg/m². After IV administration of a 100-mg/m² dose, the mean peak plasma level was 3.7 μg/mL (SD=0.8), with a corresponding AUC of 4.6 μg/mL·h (SD=0.8). Docetaxel (TAXOTERE®) plasma concentrations and AUC were found to be directly proportional to dose, although drug clearance was independent of dose or schedule of administration, which is consistent with a linear pharmacokinetic profile. Mean values for total body clearance and steady-state volume of distribution were 21 L/h² m and 113 L, respectively. Docetaxel (TAXOTERE®) is rapidly and extensively distributed following intravenous (IV) administration. In vitro studies show that it is approximately 94% bound to plasma proteins, primarily to albumin, α₁-acid glycoproteins, and lipoproteins.

The dosage schedule for TAXOTERE® (docetaxel) varies with the type of cancer it is treating. For breast cancer, the recommended dosage is 60-100 mg/m² intravenously over 1 hour every 3 weeks. In cases of non-small cell lung cancer, TAXOTERE® is used only after failure of prior platinum-based chemotherapy. The recommended dosage is 75 mg/m² intravenously over 1 hour every 3 weeks.

An important limitation associated with docetaxel use is the unpredictable interindividual variability in efficacy and toxicity. Since its clinical introduction, attempts to improve docetaxel treatment have covered various areas: reducing the interindividual pharmacokinetic (PK) and pharmacodynamic (PD) variability, optimizing schedule, route of administration and drug formulation, and reversing drug resistance.

Analogues of docetaxel have been described, including 3′-dephenyl-3′cyclohexyldocetaxel, 2-(hexahydro)docetaxel, and 3′-dephenyl-3′cyclohexyl-2-(hexahydro)docetaxel. These docetaxel analogues contain cyclohexyl groups instead of phenyl groups at the C-3′ and/or C-2 benzoate positions. Ojima et al., “Synthesis and Structure-Activity Relationships of New Antitumor Taxoids: Effects of Cyclohexyl Substitution at the C-3′ and/or C-2 TAXOTERE® (Docetaxel),” J. Med. Chem., 37:2602-08 (1994). 3′-dephenyl-3′-cyclohexyldocetaxel and 2-(hexahydro)docetaxel have been reported to possess strong inhibitory activity for microtubule disassembly equivalent to docetaxel. This demonstrates that phenyl or an aromatic group at C-3′ or C-2 is not a requisite for strong binding to the microtubules.

Other previously described docetaxel analogues include various 2-amido docetaxel analogues, including m-methoxy and m-chlorobenzoylamido analogues (Fang et al., “Synthesis and Cytotoxicity of 2alpha-amido Docetaxel Analogues,” Bioorg. Med. Chem. Lett., 12:1543-6 (2002)); docetaxel analogues lacking the oxetane D-ring but possessing the 4alpha-acetoxy group, which is important for biological activity (Deka et al., “Deletion of the oxetane ring in docetaxel analogues: synthesis and biological evaluation,” Org. Lett., 5:5031-4 (2003)); 5(20)deoxydocetaxel (Dubois et al., “Synthesis of 5(20)deoxydocetaxel, a new active docetaxel analogue,” Tetrahedron Lett., 41:3331-3334 (2000)); 10-deoxy-10-C-morpholinoethyl docetaxel analogues (limura et al., “Orally active docetaxel analogue—of 10-deoxy-10-C-morpholinoethyl docetaxel analogues,” Bioorganic and Medicinal Chem. Lett., 11:407-410 (2001)); docetaxel analogues described in Cassidy et al., Clin. Can. Res., 8:846-855 (2002), such as analogues having a t-butyl carbamate as the isoserine N-acyl substituent, but differing from docetaxel at C-10 (acetyl group versus hydroxyl) and at the C-13 isoserine linkage (enol ester versus ester); and docetaxel analogues having a peptide side chain at C3, described in Larroque et al., “Novel C2-C3 “N-peptide linked macrocyclic taxoids. Part 1: Synthesis and biological activities of docetaxel analogues with a peptide side chain at C3”, Bioorg. Med. Chem. Lett. 15(21):4722-4726 (2005). In addition, various docetaxel derivatives are in clinical trials, including XRP9881 (also referred to as RPR 109881A) (10-deacetyl baccatin III docetaxel analogue) (Aventis Pharma), XRP6528 (10-deacetyl baccatin III docetaxel analogue) (Aventis Pharma), Ortataxel (14-beta-hydroxy-deacetyl baccatin III docetaxel analogue) (Bayer/Indena), MAC-321 (10-deacetyl-7-propanoyl baccatin docetaxel analogue) (Wyeth-Ayerst), and DJ-927 (7-deoxy-9-beta-dihydro-9, 10, 0-acetal taxane docetaxal analogue) (Daiichi Pharmaceuticals), all described in Engels et al., “Potential for Improvement of Docetaxel-Based Chemotherapy: A Pharmacological Review,” British J. of Can., 93:173-177 (2005). Additional docetaxel derivatives are described in Querolle et al., “Novel C2-C3′N-linked Macrocyclic Taxoids: Novel Docetaxel Analogues with High Tubulin Activity,” J. Med. Chem., (November 2004).

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto or associated with the surface thereof a non-crosslinked surface stabilizer. The '684 patent does not describe nanoparticulate compositions of docetaxel or an analogue thereof.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for ““Method of X-Ray Imaging Using lodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,582,285 for “Apparatus for sanitary wet milling;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” and U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference. None of these patents describe nanoparticulate formulations of docetaxel or analogues thereof.

Amorphous small particle compositions are described, for example, in U.S. Pat. Nos. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

There is currently a need for docetaxel formulations that have enhanced solubility characteristics which, in turn, provide enhanced bioavailability and reduced toxicity upon administration to a patient. The present invention satisfies these needs by providing methods and compositions comprising nanoparticulate formulations of docetaxel and analogues thereof. Such formulations include, but are not limited to, injectable nanoparticulate docetaxel or analogues thereof formulations.

### SUMMARY OF THE INVENTION

The present invention relates to nanoparticulate docetaxel compositions comprising docetaxel or an analogue thereof, wherein the docetaxel or analogue thereof particles have an effective average particle size of less than about 2000 nm. The compositions also comprise at least one surface stabilizer adsorbed onto or associated with the surface of docetaxel or docetaxel analogue particles. A preferred dosage form of the invention is an injectable dosage form, although any pharmaceutically acceptable dosage form can be utilized.

Another aspect of the invention is directed to pharmaceutical compositions comprising nanoparticulate docetaxel or an analogue thereof, at least one surface stabilizer, and a pharmaceutically acceptable carrier, as well as any desired excipients.

In one embodiment of the invention, an injectable formulation of docetaxel or an analogue thereof is provided. In another embodiment, the formulation does not contain polysorbate (including Polysorbate 80) or ethanol in water.

One aspect of the invention is directed to the surprising and unexpected discovery of a new injectable formulation of docetaxel or an analogue thereof (collectively referred to as the “active ingredient”), that accomplishes the following objectives upon administration: (1) the injectable formulation does not require the presence of a polysorbate or ethanol in water and (2) the effective average particle size of the nanoparticulate docetaxel or analogue thereof is less than about 2 microns. In one embodiment, the injectable formulation comprises a nanoparticulate docetaxel or analogue thereof and a povidone polymer as a surface stabilizer adsorbed on or associated with the surface of the docetaxel or analogue thereof.

The invention provides for compositions comprising concentrations of docetaxel or analogue thereof free of polysorbate and/or ethanol in low injection volumes, with rapid drug dissolution upon administration.

Another aspect of the invention is directed to nanoparticulate compositions comprising docetaxel or an analogue thereof having improved pharmacokinetic profiles as compared to conventional docetaxel formulations, such as TAXOTERE®.

Another embodiment of the invention is directed to nanoparticulate compositions comprising docetaxel or an analogue thereof and further comprising one or more non-docetaxel or non-docetaxel analogue active agents known in the art as being useful in treating cancer or commonly used in conjunction with a taxoid.

This invention further discloses a method of making the inventive nanoparticulate compositions comprising docetaxel or an analogue thereof. Such a method comprises contacting the nanoparticulate docetaxel or analogue thereof particles with at least one surface stabilizer for a time and under conditions sufficient to provide a nanoparticulate docetaxel or analogue thereof composition having an effective average particle size of less than about 2000 nm. The one or more surface stabilizers can be contacted with docetaxel or the analogue thereof either before, during, or after size reduction of the docetaxel.

The present invention is also directed to methods of treating cancer using the novel nanoparticulate docetaxel or analogue thereof compositions disclosed herein. Such methods comprise administering to a subject a therapeutically effective amount of a nanoparticulate docetaxel or analogue thereof composition according to the invention. Other methods of treatment using the nanoparticulate compositions of the invention are known to those skilled in the art.

Both the foregoing general description and the following brief description of the drawings and detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1. Light micrograph using phase optics at 100× of unmilled docetaxel (anhydrous) (Camida Ltd.).

FIG. 2. Light micrograph using phase optics at 100 × of an aqueous nanoparticulate dispersion of 5% (w/w) docetaxel (Camidta Ltd.), combined with 1.25% (w/w) polyvinylpyrrolidone (PVP) K17 and 0.25% (w/w) sodium deoxycholate.

FIG. 3. Light micrograph using phase optics at 100× of an aqueous nanoparticulate dispersion of 5% (w/w) anhydrous docetaxel (Camida Ltd.), combined with 1.25% (w/w) Tween® 80 and 0.1% (w/w) lecithin.

FIG. 4. Light micrograph using phase optics at 100× of an aqueous nanoparticulate dispersion of 5% (w/w) anhydrous docetaxel (Camida Ltd.), combined with 1.25% (w/w) polyvinylpyrrolidone (PVP) K12, 0.25% (w/w) sodium deoxycholate, and 20% (w/w) dextrose.

FIG. 5. Light micrograph using phase optics at 100× of an aqueous nanoparticulate dispersion of 1% (w/w) anhydrous docetaxel (Camida Ltd.), combined with 0.25% (w/w) Plasdone® S630 and 0.01% (w/w) dioctylsulfosuccinate (DOSS).

FIG. 6. Light micrograph using phase optics at 100× of an aqueous nanoparticulate dispersion of 1% (w/w) anhydrous docetaxel (Camida Ltd.), combined with 0.25% (w/w) hydroxypropylmethyl cellulose (HPMC) and 0.01% (w/w) dioctylsulfosuccinate (DOSS).

FIG. 7. Light micrograph using phase optics at 100× of an aqueous nanoparticulate dispersion of 1% (w/w) anhydrous docetaxel (Camida Ltd.), combined with 0.25% (w/w) Pluronic° F127.

FIG. 8. Light micrograph using phase optics at 100 × of unmilled trihydrate docetaxel (Camida Ltd.).

FIG. 9. Light micrograph using phase optics at 100× of an aqueous nanoparticulate dispersion of 5% (w/w) trihydrate docetaxel (Camida Ltd.), combined with 1.25% (w/w) polyvinylpyrrolidone (PVP) K12 and 0.25% (w/w) sodium deoxycholate (NaDeoxycholate).

FIG. 10. Light micrograph using phase optics at 100× of an aqueous nanoparticulate dispersion of 5% (w/w) trihydrate docetaxel (Camida Ltd.), combined with 1.25% (w/w) polyvinylpyrrolidone (PVP) K17, 0.25% (w/w) sodium deoxycholate, and 20% (w/w) dextrose.

FIG. 11. Light micrograph using phase optics at 100× of an aqueous nanoparticulate dispersion of 5% (w/w) trihydrate docetaxel (Camida Ltd.), combined with 1.25% (w/w) polyvinylpyrrolidone (PVP) K17, 0.25% (w/w) sodium deoxycholate, and 20% (w/w) dextrose.

FIG. 12. Light micrograph using phase optics at 100× of an aqueous nanoparticulate dispersion of 5% (w/w) trihydrate docetaxel (Camida Ltd.), combined with 1.25% (w/w) Tween® 80, 0.1 % (w/w) lecithin, and 20% (w/w) dextrose.

FIG. 13. Light micrograph using phase optics at 100× of an aqueous nanoparticulate dispersion of 5% (w/w) trihydrate docetaxel (Camida Ltd.), combined with 1.25% (w/w) Tween® 80, 0.1% (w/w) lecithin, and 20% (w/w) dextrose.

FIG. 14. Light micrograph using phase optics at 100× of an aqueous nanoparticulate dispersion of 5% (w/w) trihydrate docetaxel (Camida Ltd.), combined with 1.25% (w/w) TPGS (Vitamin E PEG) and 0.1% (w/w) sodium deoxycholate.

FIG. 15. Light micrograph using phase optics at 100× of an aqueous nanoparticulate dispersion of 5% (w/w) trihydrate docetaxel (Camida Ltd.), combined with 1.25% (w/w) Pluronic® F 108, 0.1% (w/w) sodium deoxycholate, and 10% (w/w) dextrose (w/w).

FIG. 16. Light micrograph using phase optics at 100× of an aqueous nanoparticulate dispersion of 5% (w/w) docetaxel, combined with 1.25% (w/w) Plasdone® S630 and 0.05% (w/w) dioctylsulfosuccinate (DOSS).

FIG. 17. Light micrograph using phase optics at 100× of an aqueous nanoparticulate dispersion of 5% (w/w) docetaxel, combined with 1.25% (w/w) HPMC and 0.05% (w/w) dioctylsulfosuccinate (DOSS).

FIG. 18. Light micrograph using phase optics at 100× of an aqueous nanoparticulate dispersion of 5% (w/w) anhydrous docetaxel, combined with 1% (w/w) albumin and 0.5% (w/w) sodium deoxycholate.

FIG. 19. Light micrograph using phase optics at 100× of an aqueous nanoparticulate dispersion of 5% (w/w) trihydrate docetaxel, combined with 1% (w/w) albumin and 0.5% (w/w) sodium deoxycholate.

### DETAILED DESCRIPTION OF THE INVENTION

A. Overview

The invention is directed to compositions comprising a nanoparticulate docetaxel or analogue thereof and methods of making and using the same. In contrast to conventional formulations of docetaxel (TAXOTERE®), the nanoparticulate compositions surprisingly and unexpectedly do not require the inclusion of polysorbate or ethanol to increase the solubility of the drug.

It was surprising that nanoparticulate compositions of docetaxel or analogues thereof could be made. While previously nanoparticulate compositions of taxol were made, docetaxel has a significantly different structure than taxol. This different structure results in docetaxel having a significantly stronger activity as compared to taxol. Moreover, docetaxel acts via a different mechanism than taxol. Given the different structures of the two compounds, it was unexpected that a surface stabilizer adsorbed to or associated with the surface of docetaxel or an analogue thereof could successfully stabilize the compound at a nanoparticulate size.

The compositions comprising docetaxel or analogue thereof have an effective average particle size of less than about 2000 nm and at least one surface stabilizer. In one embodiment, described is an injectable composition comprising nanoparticulate docetaxel or analogue thereof with a povidone polymer having a molecular weight of less than about 40,000 daltons as a surface stabilizer. In another embodiment, the nanoparticulate docetaxel or analogue thereof pharmaceutical formulation has a pH of between about 6 to about 7.

In human therapy, it is important to provide a dosage form that delivers the required therapeutic amount of the active ingredient in vivo, and that renders the active ingredient bioavailable in a rapid and constant manner. Thus, described herein are various nanoparticulate docetaxel or analogue thereof formulations that satisfy this need. Two examples of nanoparticulate docetaxel or analogue thereof dosage forms are an injectable nanoparticulate dosage form and a coated nanoparticulate dosage form, such as a solid dispersion or a liquid filled capsule, although any pharmaceutically acceptable dosage form can be utilized.

The dosage forms of the invention may be provided in formulations which exhibit a variety of release profiles upon administration to a patient including, for example, an immediate release (IR) formulation, a controlled release (CR) formulation that allows once per day administration (or other suitable time period, such as once/twice/three times per week/month), and a combination of both IR and CR formulations. Because CR forms of the compositions of the invention can require only one dose per day (or one dose per suitable time period, such as weekly or monthly), such dosage forms provide the benefits of enhanced patient convenience and compliance. The mechanism of controlled-release employed in the CR form may be accomplished in a variety of ways including, but not limited to, the use of erodable formulations, diffusion-controlled formulations, and osmotically-controlled formulations.

Advantages of the nanoparticulate docetaxel or analogue thereof formulations of the invention over conventional forms of docetaxel (e.g., non-nanoparticulate or solubilized dosage forms, such as TAXOTERE® include, but are not limited to: (1) increased water solubility; (2) increased bioavailability; (3) smaller dosage form size due to enhanced bioavailability; (4) lower therapeutic dosages due to enhanced bioavailability; (5) reduced risk of unwanted side effects; (6) enhanced patient convenience and compliance; (7) higher dosages possible without adverse side effects; and (8) more effective cancer treatment. A further advantage of the injectable nanoparticulate docetaxel or analogue thereof formulations of the invention over conventional forms of injectable docetaxel (TAXOTERE®) is the elimination of the need to use a polysorbate or ethanol to increase the solubility of the drug.

The present invention also includes nanoparticulate docetaxel or analogue thereof compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

B. Definitions

The present invention is described herein using several definitions, as set forth below and throughout the application.

The term “effective average particle size of less than about 2000 nm,” as used herein means that at least 50% of the docetaxel or analogue thereof particles have a size, by weight, of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, a “stable” docetaxel or analogue thereof particle connotes, but is not limited to a docetaxel or analogue thereof with one or more of the following parameters: (1) the docetaxel or analogue thereof particles do not appreciably flocculate or agglomerate due to interparticle attractive forces or otherwise significantly increase in particle size over time; (2) the physical structure of the docetaxel or analogue thereof particles is not altered over time, such as by conversion from an amorphous phase to a crystalline phase; (3) the docetaxel or analogue thereof particles are chemically stable; and/or (4) where the docetaxel or analogue thereof has not been subject to a heating step at or above the melting point of the docetaxel or analogue thereof in the preparation of the nanoparticles of the invention.

The term “conventional” or “non-nanoparticulate” active agent or docetaxel or analogue thereof shall mean an active agent, such as docetaxel or analogue thereof, which is solubilized or which has an effective average particle size of greater than about 2000 nm. Nanoparticulate active agents as defined herein have an effective average particle size of less than about 2000 nm.

The phrase “poorly water soluble drugs” as used herein refers to drugs that have a solubility in water of less than about 30 mg/ml, less than about 20 mg/ml, less than about 10 mg/ml, or less than about 1 mg/ml.

As used herein, the phrase “therapeutically effective amount” means the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “modified release” as used herein in relation to the composition according to the invention or a coating or coating material or used in any other context means release which is not immediate release and is taken to encompass controlled release, sustained release, and delayed release.

The term “time delay” as used herein refers to the duration of time between administration of the composition and the release of docetaxel or analogue thereof from a particular component.

The term “lag time” as used herein refers to the time between delivery of active ingredient from one component and the subsequent delivery of the docetaxel or analogue thereof from another component.

C. Features of the Nanoparticulate Docetaxel Compositions

There are a number of enhanced pharmacological characteristics of the nanoparticulate docetaxel or analogue thereof compositions of the invention.

1. Increased Bioavailability

In one embodiment of the invention, the nanoparticulate docetaxel or analogue thereof formulations exhibit increased bioavailability at the same dose of the same docetaxel or analogue thereof, and require smaller doses as compared to prior conventional docetaxel formulations, such as TAXOTERE®.

A nanoparticulate docetaxel or analogue thereof dosage form requires less drug to obtain the same pharmacological effect observed with a conventional microcrystalline docetaxel dosage form (e.g., TAXOTERE®). Therefore, the nanoparticulate docetaxel or analogue thereof dosage form has an increased bioavailability as compared to the conventional microcrystalline docetaxel dosage form.

2. The Pharmacokinetic Profiles of the Docetaxel Compositions of the Invention are not Affected by the Fed or Fasted State of the Subject Ingesting the Compositions

In another embodiment of the invention described are nanoparticulate docetaxel or analogue thereof compositions, wherein the pharmacokinetic profile of the docetaxel or analogue thereof is not substantially affected by the fed or fasted state of a subject ingesting the composition. This means that there is little or no appreciable difference in the quantity of drug absorbed or the rate of drug absorption when the nanoparticulate docetaxel or analogue thereof compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance with docetaxel or an analogue thereof, an increase in the medical condition for which the drug is being prescribed may be observed—i.e., the prognosis for a cancer patient, such as a breast or lung cancer patient, may worsen.

The invention also provides docetaxel or analogue thereof compositions having a desirable pharmacokinetic profile when administered to mammalian subjects. The desirable pharmacokinetic profile of the docetaxel or analogue thereof compositions preferably includes, but is not limited to: (1) a Cₘₐₓ for docetaxel or analogue thereof, when assayed in the plasma of a mammalian subject following administration, that is greater than the Cₘₐₓ for a non-nanoparticulate docetaxel formulation (e.g., TAXOTERE®), administered at the same dosage; and/or (2) an AUC for docetaxel or analogue thereof, when assayed in the plasma of a mammalian subject following administration, that is greater than the AUC for a non-nanoparticulate docetaxel formulation (e.g., TAXOTERE®), administered at the same dosage; and/or (3) a Tₘₐₓ for docetaxel or analogue thereof, when assayed in the plasma of a mammalian subject following administration, that is less than the Tₘₐₓ for a non-nanoparticulate docetaxel formulation (e.g., TAXOTERE®, administered at the same dosage. The desirable pharmacokinetic profile, as used herein, is the pharmacokinetic profile measured after the initial dose of docetaxel or analogue thereof.

In one embodiment, a preferred docetaxel or analogue thereof composition exhibits in comparative pharmacokinetic testing with a non-nanoparticulate docetaxel formulation (e.g., TAXOTERE®, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate docetaxel formulation (e.g., TAXOTERE®).

In another embodiment, the docetaxel or analogue thereof compositions of the invention exhibit in comparative pharmacokinetic testing with a non-nanoparticulate docetaxel formulation (e.g., TAXOTERE®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate docetaxel formulation (e.g., TAXOTERE®).

In yet another embodiment, the docetaxel or analogue thereof compositions of the invention exhibit in comparative pharmacokinetic testing with a non-nanoparticulate docetaxel formulation (e.g., TAXOTERE®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate docetaxel formulation (e.g., TAXOTERE®).

3. Bioequivalency of the Docetaxel Compositions of the Invention When Administered in the Fed Versus the Fasted State

The invention also encompasses a composition comprising a nanoparticulate docetaxel or analogue thereof in which administration of the composition to a subject in a fasted state is bioequivalent to administration of the composition to a subject in a fed state.

The difference in absorption of the compositions comprising the nanoparticulate docetaxel or analogue thereof when administered in the fed versus the fasted state, is preferably less than about 100%, less than about 95%, less than about 90%, less than about 85%, less than about 80%, less than about 75%, less than about 70%, less than about 65%, less than about 60%, less than about 55%, less than about 50%, less than about 45%, less than about 35%, less than about 35%, less than about 30%, less than about 25%, less than about 20%, less than about 15%, less than about 10%, less than about 5%, or less than about 3%.

In one embodiment of the invention, the invention encompasses a nanoparticulate docetaxel or analogue thereof wherein administration of the composition to a subject in a fasted state is bioequivalent to administration of the composition to a subject in a fed state, in particular as defined by Cₘₐₓ and AUC guidelines given by the U.S. Food and Drug Administration (USFDA) and the corresponding European regulatory agency (EMEA). Under USFDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

4. Dissolution Profiles of the Docetaxel Compositions of the Invention

In yet another embodiment of the invention, the docetaxel or analogue thereof compositions of the invention have unexpectedly dramatic dissolution profiles. Rapid dissolution of docetaxel or an analogue thereof is preferable, as faster dissolution generally leads to faster onset of action and greater bioavailability. To improve the dissolution profile and bioavailability of docetaxel or an analogue thereof, it is useful to increase the drug's dissolution so that it could attain a level close to 100%.

The docetaxel or analogue thereof compositions of the invention preferably have a dissolution profile in which within about 5 minutes at least about 20% of the docetaxel or analogue thereof composition is dissolved. In other embodiments of the invention, at least about 30% or at least about 40% of the docetaxel or analogue thereof composition is dissolved within about 5 minutes. In yet other embodiments of the invention, preferably at least about 40%, at least about 50%, at least about 60%, at least about 70%, or at least about 80% of the docetaxel or analogue thereof composition is dissolved within about 10 minutes. Finally, in another embodiment of the invention, preferably at least about 70%, at least about 80%, at least about 90%, or about at least about 100% of the docetaxel or analogue thereof composition is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

5. Redispersibility Profiles of the Docetaxel Compositions of the Invention

In one embodiment of the invention, the docetaxel or analogue thereof compositions of the invention are formulated into solid dose forms which redisperse such that the effective average particle size of the redispersed docetaxel or analogue thereof particles is less than about 2 microns. This is significant, as if upon administration the nanoparticulate docetaxel or analogue thereof compositions did not redisperse to a nanoparticulate particle size, then the dosage form may lose the benefits afforded by formulating the docetaxel or analogue thereof into a nanoparticulate particle size.

Indeed, the nanoparticulate docetaxel or analogue thereof compositions of the invention benefit from the small particle size of the docetaxel or analogue thereof; if the docetaxel or analogue thereof does not redisperse into a small particle size upon administration, then “clumps” or agglomerated docetaxel or analogue thereof particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the nanoparticulate taxoid compositions of the invention, including compositions comprising a nanoparticulate docetaxel or analogue thereof exhibit dramatic redispersion of the nanoparticulate docetaxel or analogue thereof particles upon administration to a mammal, such as a human or animal, as demonstrated by reconstitution/redispersion in a biorelevant aqueous media such that the effective average particle size of the redispersed docetaxel or analogue thereof particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 N HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed docetaxel or analogue thereof particles of the invention (redispersed in an aqueous, biorelevant, or any other suitable media) have an effective average particle size of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

6. Docetaxel Compositions Used in Conjunction with Other Active Agents

The nanoparticulate docetaxel or analogue thereof compositions of the invention can additionally comprise one or more compounds useful in cancer treatment, and in particular, breast and/or lung cancer treatment. The compositions of the invention can be co-formulated with such other active agents, or the compositions of the invention can be co-administered or sequentially administered in conjunction with such active agents. Examples of drugs that can be co-administered or co-formulated with the docetaxel compositions of the invention include, but are not limited to, anticancer agents, chemotherapy agents, dexamethasone, COX-2 inhibitors, laniquidar, oblimersen, cisplatin, doxorubicin, cyclophosphamide, steroids such as prednisone and other histamine-blocking drugs, cyclophosphamide, cyclosporine, Iressa (ZD1839), thalidomide, mitoxantrone, Infliximab, erlotinib, Trastuzumab, TLK286, MDX-010, ZD1839, epirubicin, tamoxifen, bevacizumab, filgrastim, vinorelbine, cetuximab, irinotecan, estramustine, exisulind, carboplatin, ZD6474, gemcitabine, ifosfamide, capecitabine, flavopiridol, celecoxib, sulindac, and Exisulind.

D. Compositions

The invention provides compositions comprising nanoparticulate docetaxel or analogue thereof particles and at least one surface stabilizer. The surface stabilizers are preferably adsorbed onto or associated with the surface of the docetaxel or analogue thereof particles. Surface stabilizers useful herein do not chemically react with the docetaxel or analogue thereof particles or itself. Preferably, individual molecules of the surface stabilizer are essentially free of intermolecular cross-linkages. In another embodiment, the compositions of the present invention can comprise two or more surface stabilizers.

The present invention also includes nanoparticulate docetaxel or analogue thereof compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like. In certain embodiments of the invention, the nanoparticulate docetaxel or analogue thereof formulations are in an injectable form or a coated oral form.

1. Docetaxel

As used herein, the term “docetaxel” includes analogs and salts thereof, and can be in a crystalline phase, an amorphous phase, a semi-crystalline phase, a semi-amorphous phase, or a mixture thereof. Docetaxel or an analogue thereof may be present either in the form of one substantially optically pure enantiomer or as a mixture, racemic or otherwise, of enantiomers.

Analogues of docetaxel described and encompassed by the invention include, but are not limited to,

(1) docetaxel analogues comprising cyclohexyl groups instead of phenyl groups at the C-3′ and/or C-2 benzoate positions, such as 3′-dephenyl-3′cyclohexyldocetaxel, 2-(hexahydro)docetaxel, and 3′-dephenyl-3′cyclohexyl-2-(hexahydro)docetaxel (Ojima et al., “Synthesis and structure-activity relationships of new antitumor taxoids. Effects of cyclohexyl substitution at the C-3′ and/or C-2 of taxotere (docetaxel),” J. Med. Chem., 37(16):2602-8 (1994));

(2) docetaxel analogues lacking phenyl or an aromatic group at C-3′ or C-2 position, such as 3′-dephenyl-3′-cyclohexyldocetaxel and 2-(hexahydro)docetaxel;

(3) 2-amido docetaxel analogues, including m-methoxy and m-chlorobenzoylamido analogues (Fang et al., Bioorg. Med. Chem. Lett., 12(11):1543-6 (2002);

(4) docetaxel analogues lacking the oxetane D-ring but possessing the 4alpha-acetoxy group, which is important for biological activity, such as 5(20)-thia docetaxel analogues, which can be synthesized from 10-deacetylbaccatin III or taxine B and isotaxine B, described in Merckle et al., “Semisynthesis of D-ring modified taxoids: novel thia derivatives of docetaxel,” J. Org. Chem., 66(15):5058-65 (2001), and Deka et al., Org. Lett., 5(26):5031-4 (2003);

(5) 5(20)deoxydocetaxel;

(6) 10-deoxy-10-C-morpholinoethyl docetaxel analogues, including doctaxel analogues in which the 7-hydroxyl group is modified to hydrophobic groups (methoxy, deoxy, 6,7-olefin, alpha-F, 7-beta-8-beta-methano, fluoromethoxy), described in Iimura et al., “Orally active docetaxel analogue: synthesis of 10-deoxy-10-C-morpholinoethyl docetaxel analogues,” Bioorg. Med. Chem. Lett., 11(3):407-10 (2001);

(7) docetaxel analogues described in Cassidy et al., Clin. Can. Res., 8:846-855 (2002), such as analogues having a t-butyl carbamate as the isoserine N-acyl substituent, but differing from docetaxel at C-10 (acetyl group versus hydroxyl) and at the C-13 isoserine linkage (enol ester versus ester);

(8) docetaxel analogues having a peptide side chain at C3, described in Larroque et al., “Novel C2-C3 “N-peptide linked macrocyclic taxoids. Part 1: Synthesis and biological activities of docetaxel analogues with a peptide side chain at C3”, Bioorg. Med. Chem. Lett. 15(21):4722-4726 (2005);

(9) XRP9881 (10-deacetyl baccatin III docetaxel analogue);

(10) XRP6528 (10-deacetyl baccatin III docetaxel analogue);

(11) Ortataxel (14-beta-hydroxy-deacetyl baccatin III docetaxel analogue);

(12) MAC-321 (1 0-deacetyl-7-propanoyl baccatin docetaxel analogue);

(13) DJ-927 (7-deoxy-9-beta-dihydro-9,10, 0-acetal taxane docetaxal analogue);

(14) docetaxel analogues having C2-C3′N-linkages bearing an aromatic ring at position C2, and tethered between N3′ and the C2-aromatic ring at the ortho, meta, or para position. The para-substituted derivatives were unable to stabilize microtubules, whereas the ortho- and meta-substituted compounds show significant activity in cold-induced microtubule disassembly assay. Olivier et al., “Synthesis of C2-C3′N-Linked Macrocyclic Taxoids; Novel Docetaxel Analogues with High Tubulin Activity,” J. Med. Chem., 47(24:5937-44 (November 2004);

(15) docetaxel analogues bearing 22-membered (or more) rings connecting the C-2 OH and C-3′ NH moieties (biological evaluation of docetaxel analogues bearing 18-, 20-, 21-, and 22-membered rings connecting the C-2 OH and C-3′ NH moieties showed that activity is dependent on the ring size; only the 22-membered ring taxoid 3d exhibited significant tubulin binding) (Querolle et al., “Synthesis of novel macrocyclic docetaxel analogues. Influence of their macrocyclic ring size on tubulin activity,” J. Med. Chem., 46(17):3623-30 (2003).);

(16) 7beta-O-glycosylated docetaxel analogue (Anastasia et al., “Semi-Synthesis of an O-glycosylated docetaxel analogue,” Bioorg. Med. Chem., 11(7):1551-6 (2003));

(17) 1 0-alkylated docetaxel analogues, such as a 10-alkylated docetaxel analogue having a methoxycarbonyl group at the end of the alkyl moiety (Nakayama et al., “Synthesis and cytotoxic activity of novel 10-alkylated docetaxel analogs,” Bioorg. Med. Chem. Lett., 8(5):427-32 (1998));

(18) 2′,2′-difluoro, 3′-(2-furyl), and 3′-(2-pyrrolyl) docetaxel analogues (Uoto et al., “Synthesis and structure-activity relationships of novel 2′, 2′-difluoro analogues of docetaxel,” Chem. Pharm. Bull. (Tokyo), 45(11):1793-804 (1997)); and

(19) Fluorescent and biotinylated docetaxel analogues, such as docetaxel analogues that possess (a) a N-(7-nitrobenz-2-oxa-1,3-diazo-4-yl)amido-6-caproyl chain in position 7 or 3′, (b) a N-(7-nitrobenz-2-oxa-1,3-diazo-4-yl)amido-3-propanoyl group at 3′, or (c) a 5′-biotinyl amido-6-caproyl chain in position 7, 10 or 3′ (Dubois et al., “Fluorescent and biotinylated analogues of docetaxel: synthesis and biological evaluation,” Bioorg. Med. Chem., 3(10):1357-68 (1995)).

2. Surface Stabilizers

Combinations of more than one surface stabilizer can be used in the docetaxel or analogue thereof formulations of the invention. In one embodiment of the invention, the docetaxel or analogue thereof formulation is an injectable formulation. Suitable surface stabilizers include, but are not limited to, known organic and inorganic pharmaceutical excipients. Such excipients include various polymers, low molecular weight oligomers, natural products, and surfactants. Surface stabilizers include nonionic, ionic, anionic, cationic, and zwitterionic surfactants. In one embodiment of the invention, a surface stabilizer for an injectable nanoparticulate docetaxel or analogue thereof formulation is a povidone polymer.

Representative examples of surface stabilizers include hydroxypropyl methylcellulose (now known as hypromellose), albumin, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Speciality Chemicals)); polyethylene glycols (e.g., Carbowaxes 3550® and 934′ (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-IOG® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂O H)₂ (Eastman Kodak Co.); decanoyl-N-methylglucamide; n-decyl (-D-glucopyranoside; n-decyl (-D-maltopyranoside; n-dodecyl (-D-glucopyranoside; n-dodecyl (-D-maltoside; heptanoyl-N-methylglucamide; n-heptyl-(-D-glucopyranoside; n-heptyl (-D-thioglucoside; n-hexyl (-D-glucopyranoside; nonanoyl-N-methylglucamide; n-noyl (-D-glucopyranoside; octanoyl-N-methylglucamide; n-octyl-(-D-glucopyranoside; octyl (-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like. Also, if desirable, the nanoparticulate docetaxel or analogue thereof formulations of the present invention can be formulated to be phospholipid-free.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate. Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulfate, lauryl dimethyl benzyl ammonium chloride or bromide, lauryl dimethyl (ethenoxy)4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336), POLYQUAT, tetrabutylammonium bromide, benzyl trimethylammonium bromide, choline esters (such as choline esters of fatty acids), benzalkonium chloride, stearalkonium chloride compounds (such as stearyltrimonium chloride and distearyldimonium chloride), cetyl pyridinium bromide or chloride, halide salts of quaternized polyoxyethylalkylamines, MIRAPOL and ALKAQUAT (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR1R2R3R4(+). For compounds of the formula NR1R2R3R4(+):
(i) none of R1-R4 are CH3;
(ii) one of R1-R4 is CH3;
(iii) three of R1-R4 are CH3;
(iv) all of R1-R4 are CH3;
(v) two of R1-R4 are CH3, one of R1-R4 is C6H5CH2, and one of R1-R4 is an alkyl chain of seven carbon atoms or less;
(vi) two of R1-R4 are CH3, one of R1-R4 is C6H5CH2, and one of R1-R4 is an alkyl chain of nineteen carbon atoms or more;
(vii) two of R1-R4 are CH3 and one of R1-R4 is the group C6H5(CH2)n, where n>1;
(viii) two of R1-R4 are CH3, one of R1-R4 is C6H5CH2, and one of R1-R4 comprises at least one heteroatom;
(ix) two of R1-R4 are CH3, one of R1-R4 is C6H5CH2, and one of R1-R4 comprises at least one halogen;
(x) two of R1-R4 are CH3, one of R1-R4 is C6H5CH2, and one of R1-R4 comprises at least one cyclic fragment;
(xi) two of R1-R4 are CH3 and one of R1-R4 is a phenyl ring; or
(xii) two of R1-R4 are CH3 and two of R1-R4 are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCI, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated herein by reference.

Povidone Polymers

Povidone polymers are exemplary surface stabilizers for use in formulating an injectable nanoparticulate docetaxel or analogue thereof formulation. Povidone polymers, also known as polyvidon(e), povidonum, PVP, and polyvinylpyrrolidone, are sold under the trade names Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. To be useful as a surface stabilizer for injectable nanoparticulate docetaxel or analogue thereof compositions, it is preferable that the povidone polymer have a molecular weight of less than about 40,000 daltons, as a molecular weight of greater than 40,000 daltons would have difficulty clearing the body for injectables.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° C. to form γ-butyrolactone; and (3) reacting y-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10^{th} Edition, pp. 7581 (Merck & Co., Rahway, N.J., 1983).

The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering. Table 1 provides molecular weight data for several commercially available povidone polymers, all of which are soluble.

While applicants do not wish to be bound by theorectical mechanisms, it is believed that the povidone polymer hinders the flocculation and/or agglomeration of the particles of the docetaxel or analogue thereof by functioning as a mechanical or steric barrier between the particles, minimizing the close, interparticle approach necessary for agglomeration and flocculation.

TABLE 1

| Povidone | K-Value | Mv (Daltons)** | Mw (Daltons)** | Mn (Daltons)** |
|---|---|---|---|---|
| Plasdone ® C-15 | 17 ± 1 | 7,000 | 10,500 | 3,000 |
| Plasdone ® C-30 | 30.5 ± 1.5 | 38,000 | 62,500* | 16,500 |
| Kollidon ® 12 PF | 11-14 | 3,900 | 2,000-3,000 | 1,300 |
| Kollidon ® 17 PF | 16-18 | 9,300 | 7,000-11,000 | 2,500 |
| Kollidon ® 25 | 24-32 | 25,700 | 28,000-34,000 | 6,000 |

*Because the molecular weight is greater than 40,000 daltons, this povidone polymer is not useful as a surface stabilizer for a drug compound to be administered parenterally (i.e., injected).
**Mv is the viscosity-average molecular weight, Mn is the number-average molecular weight, and Mw is the weight average molecular weight. Mw and Mn were determined by light scattering and ultra-centrifugation, and Mv was determined by viscosity measurements.

Based on the data provided in Table 1, exemplary preferred commercially available povidone polymers for injectable compositions include, but are not limited to, Plasdone® C-5, Kollidon® 12 PF, Kollidon® 17 PF, and Kollidon®25.

3. Nanoparticulate Docetaxel Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

Compositions of the invention comprise docetaxel or an analogue thereof particles having an effective average particle size of less than about 2 microns. In other embodiments of the invention, the docetaxel or analogue thereof particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 run, as measured by light-scattering methods, microscopy, or other appropriate methods. In another embodiment of the invention, the compositions of the invention are in an injectable dosage form and the docetaxel or analogue thereof particles preferably have an effective average particle size of less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Injectable compositions can comprise docetaxel or an analogue thereof having an effective average particle size of greater than about 1 micron, up to about 2 microns.

An “effective average particle size of less than about 2000 nm” means that at least 50% of the docetaxel or analogue thereof particles have a particle size less than the effective average, by weight, i.e., less than about 2000 nm. If the “effective average particle size” is less than about 600 nm, then at least about 50% of the docetaxel or analogue thereof particles have a size of less than about 600 nm, when measured by the above-noted techniques. The same is true for the other particle sizes referenced above.

In other embodiments, at least about 60%, at least about 70%, at least about at least about 80%, at least about 90%, at least about 95%, or at least about 99% of the docetaxel or analogue thereof particles have a particle size less than the effective average, i.e., less than about 1000 nm, about 900 nm, about 800 nm, etc..

In the invention, the value for D50 of a nanoparticulate docetaxel or analogue thereof composition is the particle size below which 50% of the docetaxel or analogue thereof particles fall, by weight. Similarly, D90 is the particle size below which 90% of the docetaxel or analogue thereof particles fall, by weight.

4. Concentration of Nanoparticulate Docetaxel and Surface Stabilizers

The relative amounts of docetaxel or analogue thereof and one or more surface stabilizers can vary widely. The optimal amount of the individual components depends, for example, upon physical and chemical attributes of the surface stabilizer(s) and docetaxel or analogue thereof selected, such as the hydrophilic lipophilic balance (HLB), melting point, and the surface tension of water solutions of the stabilizer, etc.

Preferably, the concentration of the docetaxel or analogue thereof can vary from about 99.5% to about 0.001%, from about 95% to about 0.1%, or from about 90% to about 0.5%, by weight, based on the total combined weight of the docetaxel or analogue thereof and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of the docetaxel or analogue thereof and at least one surface stabilizer, not including other excipients.

5. Other Pharmaceutical Excipients

Pharmaceutical compositions of the invention may also comprise one or more binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are well known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, and quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples, such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

6. Injectable Nanoparticulate Docetaxel Formulations

In one embodiment of the invention, provided are injectable nanoparticulate docetaxel or analogue thereof formulations that can comprise high concentrations in low injection volumes, with rapid dissolution upon administration. Exemplary compositions comprise, based on % w/w:

| | |
|---|---|
| Docetaxel or analogue | 5-50% |
| Surface stabilizer | 0.1-50% |
| preservatives | 0.05-0.25% |
| pH adjusting agent | pH about 6 to about 7 |
| water for injection | q.s. |

Exemplary preservatives include methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

7. Coated Oral Formulations

Docetaxel or analogue thereof bioavailability is reduced when administered with food. Administration with food causes an increase in the amount of time that the docetaxel or analogue thereof is retained in the stomach. This increased retention time allows the docetaxel or analogue thereof to dissolve in the acidic stomach conditions. Then, when the dissolved drug exits the stomach and enters the more basic conditions of the upper small intestine, the docetaxel or analogue thereof precipitates out of solution. The precipitated docetaxel or analogue thereof is poorly absorbed since it must once again dissolve before it can be absorbed and this process is slow because of the poor water solubility of docetaxel or analogue thereof. Dissolution of the drug in the stomach, followed by precipitation, diminishes the enhanced bioavailability that docetaxel or analogue thereof can gain from administration as a nanoparticulate dosage form, such as nanoparticulate docetaxel or analogue thereof solid dispersion, or nanoparticulate docetaxel or analogue thereof liquid filled capsule. Protection of the drug from the low pH conditions of the stomach would reduce or eliminate this decrease in bioavailability.

Therefore, a composition comprising coated nanoparticulate docetaxel or analogue thereof, such as an enteric coated docetaxel or analogue thereof is described herein. In one embodiment, the oral formulation comprises an oral formulation, such as an enteric coated solid dosage form.

Solid dosage forms for oral administration include, but are not limited to, capsules, tablets, pills, powders, and granules. In such solid dosage forms, the docetaxel or analogue thereof is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents Drug Release Profiles

In one embodiment, the coated docetaxel or analogue thereof, such as the enteric-coated docetaxel or analogue thereof composition described herein exhibits a pulsatile plasma profile when administered to a patient in an oral dosage form. The plasma profile associated with the administration of a drug compound may be described as a “pulsatile profile” in which pulses of high docetaxel or analogue thereof concentration, interspersed with low concentration troughs, are observed. A pulsatile profile containing two peaks may be described as “bimodal”. Similarly, a composition or a dosage form which produces such a profile upon administration may be said to exhibit “pulsed release” of the docetaxel or analogue thereof.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of a docetaxel or analogue thereof between peaks has been thought to be a contributing factor in reducing or preventing patient tolerance to various types of drugs.

Multiparticulate modified controlled release (CR) compositions similar to those disclosed herein are disclosed and claimed in the U.S. Pat. Nos. 6,228,398, 6,730,325 and 6,793,936 to Devane et al; all of which are specifically incorporated by reference herein. All of the relevant prior art in this field may be found therein.

Another aspect of the present invention is a multiparticulate modified release composition having a first component comprising a first population of the docetaxel or analogue thereof and a second component comprising a second population of the docetaxel or analogue thereof. The ingredient-containing particles of the second component are coated with a modified release coating. Alternatively or additionally, the second population of the docetaxel or analogue thereof -containing particles further comprises a modified release matrix material. Following oral delivery, the composition in operation delivers the docetaxel or analogue thereof in a pulsatile manner.

In a preferred embodiment of a multiparticulate modified release composition according to the invention, the first component is an immediate release component.

The modified release coating applied to the second population of the docetaxel or analogue thereof particles causes a lag time between the release of active from the first population of the docetaxel or analogue thereof-containing particles and the release of active from the second population of active docetaxel or analogue thereof-containing particles. Similarly, the presence of a modified release matrix material in the second population of the docetaxel or analogue thereof -containing particles causes a lag time between the release of the docetaxel or analogue thereof from the first population of the docetaxel or analogue thereof -containing particles and the release of active ingredient from the second population of the docetaxel or analogue thereof -containing particles. The duration of the lag time may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the multiparticulate modified release composition upon administration is substantially similar to the plasma profile produced by the administration of two or more IR dosage forms given sequentially, the multiparticulate controlled release composition of the present invention is particularly useful for administering docetaxel or analogue thereof for which patient tolerance may be problematical. This multiparticulate modified release composition is therefore advantageous for reducing or minimizing the development of patient tolerance to the active ingredient in the composition.

The present invention further provides a method for treating cancer, in particular breast, ovarian, prostate, and/or lung cancer, comprising administering a therapeutically effective amount of a composition according to the invention to provide pulsed or bimodal administration of a docetaxel or analogue thereof. Advantages of the invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the compositions of the invention would contribute to reducing health care costs by reducing the amount of time spent by health care workers on the administration of drugs.

The active ingredient in each component may be the same or different. For example, a composition in which the first component contains docetaxel or analogue thereof and the second component comprises a second active ingredient may be desirable for combination therapies. Indeed, two or more active ingredients may be incorporated into the same component when the active ingredients are compatible with each other. A drug compound present in one component of the composition may be accompanied by, for example, an enhancer compound or a sensitizer compound in another component of the composition, to modify the bioavailability or therapeutic effect of the drug compound.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

The proportion of the docetaxel or analogue thereof present in each component may be the same or different depending on the desired dosing regime. The docetaxel or analogue thereof is present in the first component and in the second component in any amount sufficient to elicit a therapeutic response. The docetaxel or analogue thereof when applicable, may be present either in the form of one substantially optically pure enantiomer or as a mixture, racemic or otherwise, of enantiomers.

The time-release characteristics for the release of the docetaxel or analogue thereof from each of the components may be varied by modifying the composition of each component, including modifying any of the excipients or coatings which may be present. In particular the release of the docetaxel or analogue thereof may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of the docetaxel or analogue thereof from each component may also be varied by modifying the composition of each of the components, including modifying any excipients and coatings which may be present. For example, the first component may be an immediate release component wherein the docetaxel or analogue thereof is released substantially immediately upon administration. Alternatively, the first component may be, for example, a time-delayed immediate release component in which the docetaxel or analogue thereof is released substantially immediately after a time delay. The second component may be, for example, a time-delayed immediate release component as just described or, alternatively, a time-delayed sustained release or extended release component in which the docetaxel or analogue thereof is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of the docetaxel or analogue thereof in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of the docetaxel or analogue thereof from each component and the nature of the release from each component (i.e. immediate release, sustained release etc.), the pulses in the plasma profile may be well separated and clearly defined peaks (e.g. when the lag time is long) or the pulses may be superimposed to a degree (e.g. in when the lag time is short).

In a preferred embodiment, the multiparticulate modified release composition according to the present invention has an immediate release component and at least one modified release component, the immediate release component comprising a first population of the docetaxel or analogue thereof-containing particles and the modified release components comprising second and subsequent populations of the docetaxel or analogue thereof-containing particles. The second and subsequent modified release components may comprise a controlled release coating. Additionally or alternatively, the second and subsequent modified release components may comprise a modified release matrix material. In operation, administration of such a multiparticulate modified release composition having, for example, a single modified release component results in characteristic pulsatile plasma concentration levels of the docetaxel or analogue thereof in which the immediate release component of the composition gives rise to a first peak in the plasma profile and the modified release component gives rise to a second peak in the plasma profile. Embodiments of the invention comprising more than one modified release component give rise to further peaks in the plasma profile.

Such a plasma profile produced from the administration of a single dosage unit is advantageous when it is desirable to deliver two (or more) pulses of docetaxel or analogue thereof without the need for administration of two (or more) dosage units.

Enteric Coating

Any coating material which modifies the release of the docetaxel or analogue thereof in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the Trade Mark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, Polyox polyethylene oxides (m. wt. about 100 k-5,000 k), AquaKeep acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of docetaxel or analogue thereof dispersed therein in vitro or in vivo. Modified release matrix materials suitable for the practice of the present invention include but are not limited to microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acteate trimellitate, polyvinylacetate phthalate, polyalkylmethacrylates, polyvinyl acetate and mixture thereof.

A multiparticulate modified release composition according to the present invention may be incorporated into any suitable dosage form which facilitates release of the active ingredient in a pulsatile manner. Typically, the dosage form may be a blend of the different populations of docetaxel or analogue thereof-containing particles which make up the immediate release and the modified release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multi-layer tablet. In this instance the first component of the multiparticulate modified release composition may be compressed into one layer, with the second component being subsequently added as a second layer of the multi-layer tablet. The populations of docetaxel or analogue thereof-containing particles making up the composition of the invention may further be included in rapidly dissolving dosage forms such as an effervescent dosage form or a fast-melt dosage form.

In another embodiment, the composition according to the invention comprises at least two populations of docetaxel or analogue thereof -containing particles which have different in vitro dissolution profiles.

Preferably, in operation the composition of the invention and the solid oral dosage forms containing the composition release the docetaxel or analogue thereof such that substantially all of the docetaxel or analogue thereof contained in the first component is released prior to release of the docetaxel or analogue thereof from the second component. When the first component comprises an IR component, for example, it is preferable that release of the docetaxel or analogue thereof from the second component is delayed until substantially all the docetaxel or analogue thereof in the IR component has been released. Release of the docetaxel or analogue thereof from the second component may be delayed as detailed above by the use of a modified release coating and/or a modified release matrix material.

In one embodiment, when it is desirable to minimize patient tolerance by providing a dosage regime which facilitates wash-out of a first dose of docetaxel or analogue thereof from a patient's system, release of the docetaxel or analogue thereof from the second component is delayed until substantially all of the docetaxel or analogue thereof contained in the first component has been released, and further delayed until at least a portion of the docetaxel or analogue thereof released from the first component has been cleared from the patient's system. In a particular embodiment, release of the docetaxel or analogue thereof from the second component of the composition in operation is substantially, if not completely, delayed for a period of at least about two hours after administration of the composition.

The release of the drug from the second component of the composition in operation is substantially, if not completely, delayed for a period of at least about four hours, preferably about four hours, after administration of the composition.

E. Methods of Making Nanoparticulate Docetaxel Compositions

Nanoparticulate docetaxel or analogue thereof compositions can be made using any suitable method known in the art such as, for example, milling, homogenization, precipitation, or supercritical fluid particle generation techniques. Exemplary methods of making nanoparticulate compositions are described in U.S. Pat. No. 5,145,684. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated herein by reference.

The resultant nanoparticulate docetaxel or analogue thereof compositions or dispersions can be utilized in solid, semi-solid, or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

An exemplary milling or homogenization method comprises: (1) dispersing docetaxel or analogue thereof in a liquid dispersion media; and (2) mechanically reducing the particle size of the docetaxel or analogue thereof to an effective average particle size of less than about 2000 nm. A surface stabilizer is added either before, during, or after particle size reduction. The pH of the liquid dispersion media is preferably maintained within the range of from about 5.0 to about 7.5 during the size reduction process. Preferably, the dispersion medium used for the size reduction process is aqueous, although any media in which the docetaxel or analogue thereof is poorly soluble and dispersible can be utilized. Examples of non-aqueous dispersion media include, but are not limited to, safflower oil, ethanol, t-butanol, glycerin, polyethylene glycol (PEG), hexane, or glycol.

Effective methods of providing mechanical force for particle size reduction of the docetaxel or analogue thereof include ball milling, media milling, and homogenization, for example, with a Microfluidizer® machine (Microfluidics Corp.). Ball milling is a low energy milling process that uses milling media, drug, stabilizer, and liquid. The materials are placed in a milling vessel that is rotated at optimal speed such that the media cascades and reduces the particle size by impaction. The media used must have a high density as the energy for the particle reduction is provided by gravity and the mass of the attrition media.

Media milling is a high energy milling process. Docetaxel or an analogue thereof, surface stabilizer, and liquid are placed in a reservoir and recirculated in a chamber containing media and a rotating shaft/impeller. The rotating shaft agitates the media, which subjects the docetaxel or analogue thereof and surface stabilizer to impaction and sheer forces, thereby reducing their size.

Homogenization is a technique that does not use milling media. Docetaxel or an analogue thereof, surface stabilizer, and liquid (or Docetaxel or an analogue thereof and liquid with the surface stabilizer added after particle size reduction) are stream propelled into a process zone, which in the Microfluidizerg machine is called the Interaction Chamber. The product to be treated is inducted into the pump, and then forced out. The priming valve of the Microfluidizer^{\dot{o}} machine purges air out of the pump. Once the pump is filled with product, the priming valve is closed and the product is forced through the interaction chamber. The geometry of the interaction chamber produces powerful forces of sheer, impact, and cavitation, which are responsible for docetaxel or an analogue thereof particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® machine also provides a heat exchanger to allow cooling of the product. U.S. Pat. No. 5,510,118 to Bosch et al., which is specifically incorporated by reference, refers to a process using a Microfluidizer® resulting in sub 400 nm particles.

Published International Patent Application No. WO 97/144407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface stabilizers.

Using a particle size reduction method, the particle size of the docetaxel or an analogue thereof is reduced to an effective average particle size of less than about 2000 nm.

The docetaxel or analogue thereof can be added to a liquid mediain which it is essentially insoluble to form a premix. The concentration of the docetaxel or an analogue thereof in the liquid media can vary from about 5 to about 60%, about 15 to about 50% (w/v), or about 20 to about 40%. The surface stabilizer can be present in the premix or it can be added to the dispersion of the docetaxel or an analogue thereof following particle size reduction. The concentration of the surface stabilizer can vary from about 0.1 to about 50%, about 0.5 to about 20%, or about 1 to about 10%, by weight.

The premix can be used directly by subjecting it to mechanical means to reduce the average particle size of the docetaxel or an analogue thereof in the dispersion to less than about 2000 nm. It is preferred that the premix be used directly when a ball mill is used for attrition. Alternatively, the docetaxel or an analogue thereof and the surface stabilizer can be dispersed in the liquid media using suitable agitation, e.g., a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the particle size of the docetaxel or an analogue thereof conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1,000 centipoise, and for ball milling the apparent viscosity of the premix is preferably from about 1 up to about 100 centipoise. Such ranges tend to afford an optimal balance between efficient particle size reduction and media erosion.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The particles of the docetaxel or an analogue thereof can be reduced in size at a temperature which does not significantly degrade it. Processing temperatures of less than about 30° C. to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of material for the grinding media is not believed to be critical. Zirconium oxide, such as 95% ZrO stabilized with magnesia, zirconium silicate, ceramic, stainless steel, titania, alumina, 95% ZrO stabilized with yttrium, and glass grinding media are exemplary grinding materials.

The grinding media can comprise particles that are preferably substantially spherical in shape, e.g., beads, consisting essentially of polymeric resin. Alternatively, the grinding media can comprise a core having a coating of a polymeric resin adhered thereon. In one embodiment of the invention, the polymeric resin can have a density from about 0.8 to about 3.0 g/cm³.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin^{\dot{o}} (E.I. du Pont de Nemours and Co.); vinyl chloride polymers and copolymers; polyurethanes; polyamides; poly(tetrafluoroethylenes), e.g., Teflon^{\dot{o}}(E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably, from about 0.03 to about 1 mm in size.

In a preferred grinding process the docetaxel or analogue thereof particles are made continuously. Such a method comprises continuously introducing docetaxel or analogue thereof into a milling chamber, contacting docetaxel or analogue thereof with grinding media while in the chamber to reduce the particle size, and continuously removing the nanoparticulate docetaxel or analogue thereof active from the milling chamber.

The grinding media is separated from the milled nanoparticulate docetaxel or analogue thereof using conventional separation techniques, in a secondary process such as by simple filtration, sieving through a mesh filter or screen, and the like. Other separation techniques such as centrifugation may also be employed.

Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

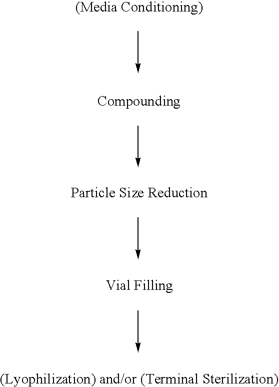

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

F. Method of Treatment

In human therapy, it is important to provide a docetaxel or analogue thereof dosage form that delivers the required therapeutic amount of the drug in vivo, and that renders the drug bioavailable in a constant manner. Thus, another aspect of the present invention provides a method of treating a mammal, including a human, requiring anti-cancer treatment including anti-tumor and anti-leukemia treatment comprising administering to the mammal the nanoparticulate docetaxel or analogue thereof formulation of the invention.

Exemplary types of cancer that can be treated with the nanoparticulate docetaxel or analogue thereof compositions of invention include, but are not limited to, breast, lung (including but not limited to non small cell lung cancer), ovarian, prostate, solid tumors (including but not limited to head and neck, breast, lung, gastrointestinal, genitourinary, melanoma, and sarcoma), primary CNS neoplasms, multiple myeloma, Non-Hodgkin's lymphoma, anaplastic astrocytoma, anaplastic meningioma, anaplastic oligodendroglioma, brain malignant hemangiopericytoma, squamous cell carcinoma of the hypopharynx, squamous cell carcinoma of the larynx, leukemia, squamous cell carcinoma of the lip and oral cavity, squamous cell carcinoma of the nasopharynx, squamous cell carcinoma of the oropharynx, cervical cancer, and pancreatic cancer.

In one embodiment of the invention, the effective dosage for the nanoparticulate docetaxel or analogue thereof compositions of the invention is less than that required for the comparable non-nanoparticulate docetaxel formulation, e.g., TAXOTERE®. The dosage schedule for TAXOTERE® (docetaxel), which is available in 20 mg (0.5 mL) and 80 mg (2.0 mL) vials, varies with the type of cancer it is treating. For breast cancer, the recommended dosage is 60-100 mg/m² intravenously over 1 hour every 3 weeks. In cases of non-small cell lung cancer, TAXOTERE® is used only after failure of prior platinum-based chemotherapy. The recommended dosage is 75 mg/m² intravenously over 1 hour every 3 weeks. Thus, in one embodiment of the invention, the dosage of the nanoparticulate docetaxel or analogue thereof compositions of the invention is less than about 100 mg/m , less than about 90 mg/m², less than about 80 mg/m², less than about 70 mg/m², less than about 60 mg/m², less than about 50 mg/m², less than about 40 mg/M², less than about 30 mg/m², less than about 20 mg/m², or less than about 10 mg/m².

In yet another embodiment of the invention, the nanoparticulate docetaxel or analogue thereof compositions of the invention can be administered at significantly higher doses as compared to the comparable non-nanoparticulate docetaxel formulation, e.g., TAXOTERE®. As described in Example 16, below, exemplary nanoparticulate docetaxel formulations exhibited a maximum in vivo tolerated dose of 500 mg/kg, in contrast to the maximum tolerated dose for TAXOTERE® of 40 mg/kg. Thus, in another embodiment of the invention, the dosage of the nanoparticulate docetaxel or analogue thereof compositions of the invention is greater than about 50 mg/m², greater than about 60 mg/², greater than about 70 mg/m², greater than about 80 mg/m², greater than about 90 mg/m², greater than about 100 mg/m², greater than about 110 mg/m², greater than about 120 mg/m², greater than about 130 mg/m², greater than about 140 mg/m², greater than about 150 mg/m², greater than about 160 mg/m², greater than about 170 mg/m², greater than about 180 mg/m², greater than about 190 mg/m², greater than about 200 mg/m², greater than about 210 mg/m², greater than about 220 mg/m², greater than about 230 mg/m², greater than about 240 mg/m², greater than about 250 mg/m², greater than about 260 mg/m², greater than about 270 mg/m², greater than about 280 mg/mM², greater than about 290 mg/mr², greater than about 300 mg/m², greater than about 310 mg/m², greater than about 320 mg/m², greater than about 330 mg/m², greater than about 340 mg/m², or greater than about 350 mg/m².

Particularly advantageous features of the invention include that the pharmaceutical formulation of the invention exhibits unexpectedly rapid absorption of the active ingredient upon administration. In one embodiment of the invention, the nanoparticulate docetaxel or analogue thereof composition, including an injectable composition, is free of polysorbate, ethanol, or a combination thereof. In addition, when formulated into an injectable formulation, the compositions of the invention can provide a high concentration in a small volume to be injected. Injectable docetaxel or analogue thereof compositions of the invention can be administered in a bolus injection or with a slow infusion over a suitable period of time.

One of ordinary skill will appreciate that effective amounts of a docetaxel or analogue thereof can be determined empirically and can be employed in pure form or, where such forms exist, in pharmaceutically acceptable salt, ester, or prodrug form. Actual dosage levels of docetaxel or analogue thereof in the injectable and oral compositions of the invention may be varied to obtain an amount of docetaxel or analogue thereof that is effective to obtain a desired therapeutic response for a particular composition and method of administration. The selected dosage level therefore depends upon the desired therapeutic effect, the route of administration, the potency of the administered docetaxel or analogue thereof, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXAMPLES

### Example 1

The purpose of this example was to prepare a nanoparticulate anhydrous docetaxel formulation.

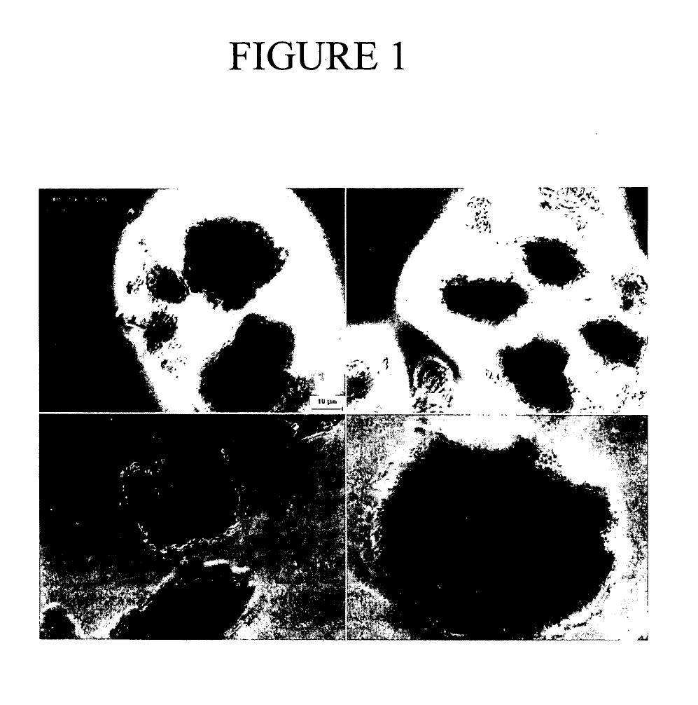

FIG. 1 shows a light micrograph of unmilled docetaxel (anhydrous) (Camida Ltd.), showing that the mean particle size of conventional, non-nanoparticulate docetaxel (anhydrous) is 212,060 nm, with a D50 of 175,530 nm and a D90 of 435,810 nm.

An aqueous dispersion of 5% (w/w) docetaxel (Camida Ltd.) was combined with 1.25% (w/w) polyvinylpyrrolidone (PVP) K17 and 0.25% (w/w) sodium deoxycholate. This mixture was then added to a 10 ml chamber of a NanoMill® 0.01 (NanoMill Systems, King of Prussia, Pa.; see e.g., U.S. Pat. No. 6,431,478), along with 220 micron PolyMill® attrition media (Dow Chemical) (89% media load). The mixture was milled at a speed of 2500 rpms for 180 minutes.

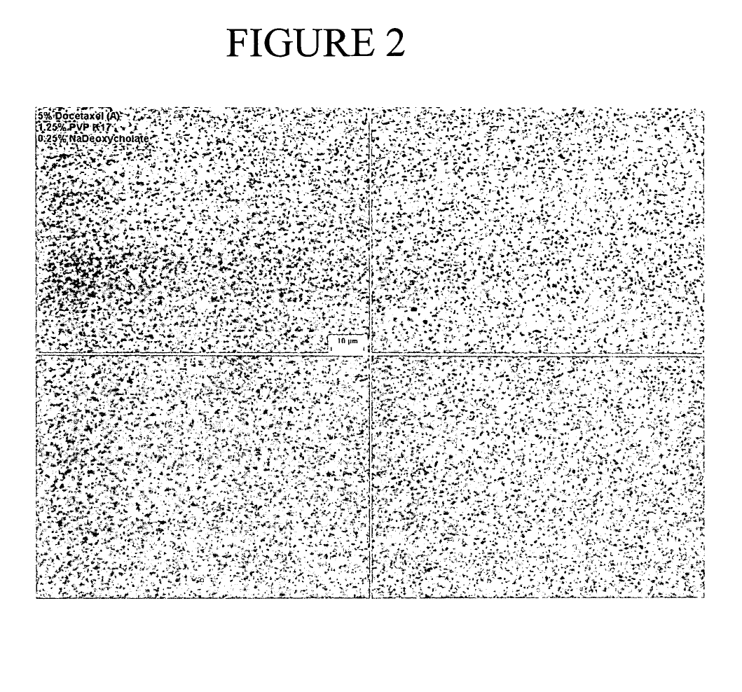

Following milling, the particle size of the milled docetaxel particles was measured, in deionized distilled water, using a Horiba LA 910 particle size analyzer. The mean milled docetaxel particle size was 170 nm, with a D50 of 145 nm and a D90 of 260 nm. FIG. 2 shows a light micrograph of the milled doectaxel.

The results demonstrate the successful preparation of a stable nanoparticulate docetaxel formulation, as the mean particle size obtained was 170 nm.

### Example 2

The purpose of this example was to prepare a nanoparticulate anhydrous docetaxel formulation.

An aqueous dispersion of 5% (w/w) anhydrous docetaxel was combined with 1.25% (w/w) Tween® 80 and 0.1% (w/w) lecithin. This mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 5500 rpms for 60 minutes.

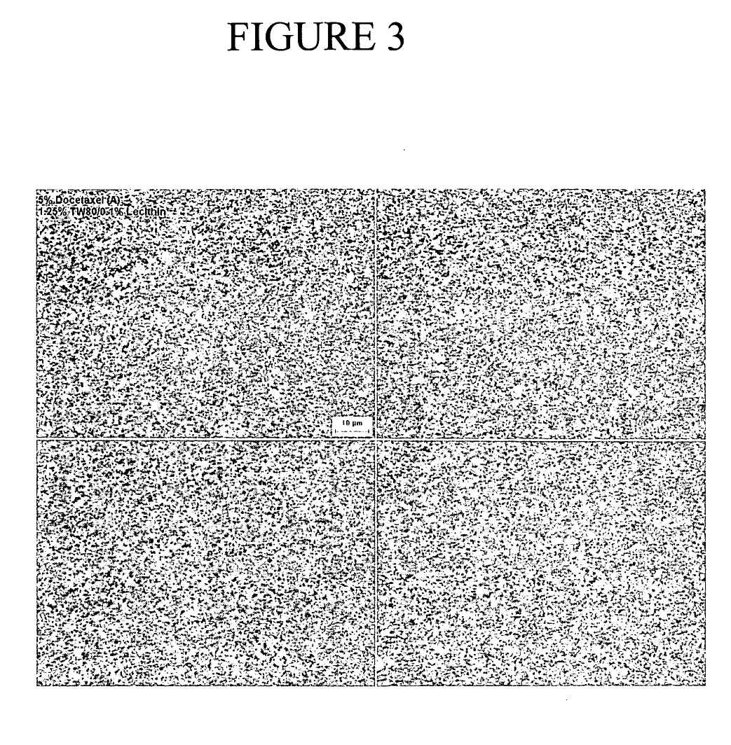

Following milling, the particle size of the milled docetaxel particles was measured, in deionized distilled water, using a Horiba LA 910 particle size analyzer. The mean milled docetaxel particle size was 166 nm, with a D50 of 147 nm and a D90 of 242 nm. FIG. 3 shows a light micrograph of the milled doectaxel.

The results demonstrate the successful preparation of a stable nanoparticulate docetaxel formulation, as the mean particle size obtained was 166 nm.

### Example 3

The purpose of this example was to prepare a nanoparticulate anhydrous docetaxel formulation.

An aqueous dispersion of 5% (w/w) anhydrous docetaxel was combined with 1.25% (w/w) polyvinylpyrrolidone (PVP) K12, 0.25% (w/w) sodium deoxycholate (w/w), and 20% (w/w) dextrose. This mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 5500 rpms for 60 minutes.

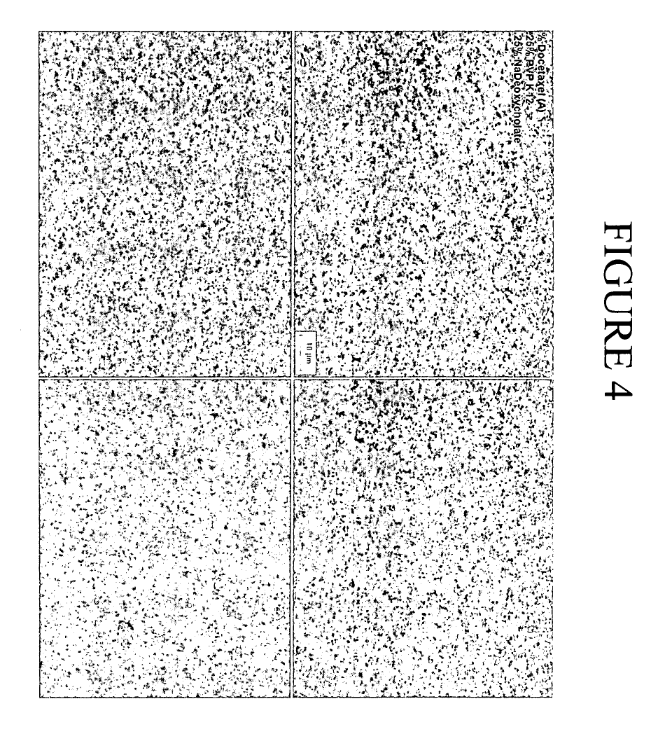

Following milling, the particle size of the milled docetaxel particles was measured, in deionized distilled water, using a Horiba LA 910 particle size analyzer. The mean milled docetaxel particle size was 165 nm, with a D50 of 142 nm and a D90 of 248 nm. FIG. 4 shows a light micrograph of the milled doectaxel.

The results demonstrate the successful preparation of a stable nanoparticulate docetaxel formulation, as the mean particle size obtained was 165 nm.

### Example 4

The purpose of this example was to prepare a nanoparticulate anhydrous docetaxel formulation.

An aqueous dispersion of 1% (w/w) anhydrous docetaxel was combined with 0.25% (w/w) Plasdone® S630 and 0.01% (w/w) dioctylsulfosuccinate (DOSS). This mixture was then milled in a 15 mL bottle using a low energy roller mill (U.S. Stoneware, Mahwah, N.J.), along with 0.5 mm ceramic media (Tosoh, Ceramics Division) (50% media load). The mixture was milled at a speed of 130 rpms for 72 hours.

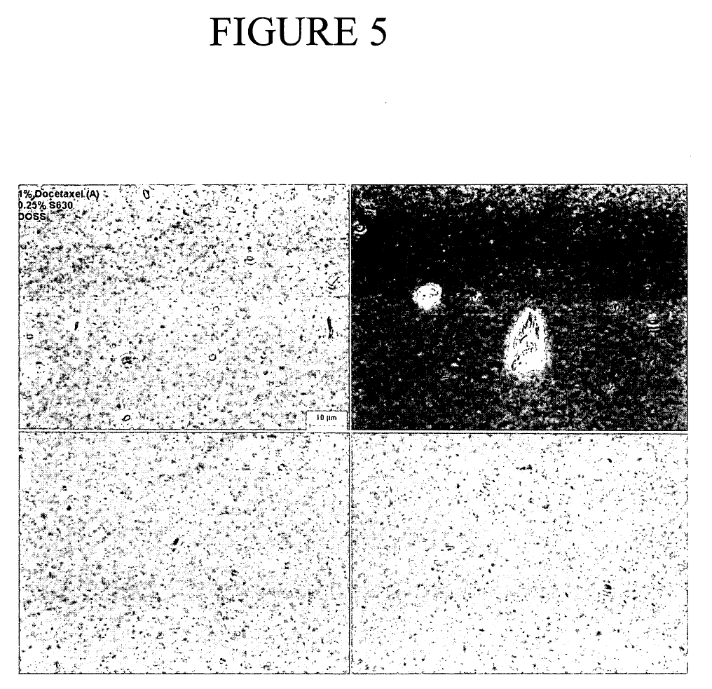

Following milling, the particle size of the milled docetaxel particles was measured, in deionized distilled water, using a Coulter N4M particle size analyzer). The mean milled docetaxel particle size was 209 nm. FIG. 5 shows a light micrograph of the milled doectaxel.

The results demonstrate the successful preparation of a stable nanoparticulate docetaxel formulation, as the mean particle size obtained was 209 nm.

### Example 5

The purpose of this example was to prepare a nanoparticulate anhydrous docetaxel formulation.

An aqueous dispersion of 1% (w/w) anhydrous docetaxel was combined with 0.25% (w/w) hydroxypropylmethyl cellulose (HPMC) and 0.01% (w/w) dioctylsulfosuccinate (DOSS). The mixture was then milled in a 15 mL glass bottle using a low energy roller mill (U.S. Stoneware, Mahwah, N.J.), along with 0.5 mm ceramic media (Tosoh, Ceramics Division) (50% media load). The mixture was milled at a speed of 130 rpms for 72 hours.

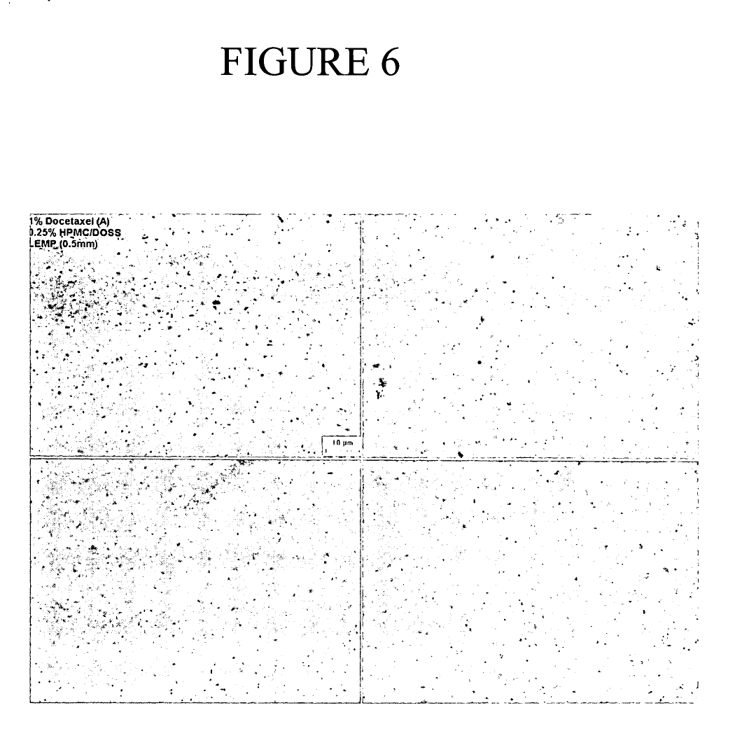

Following milling, the particle size of the milled docetaxel particles was measured, in deionized distilled water, using a Coulter N4M particle size analyzer. The mean milled docetaxel particle size was 253 nm. FIG. 6 shows a light micrograph of the milled doectaxel.

The results demonstrate the successful preparation of a stable nanoparticulate docetaxel formulation, as the mean particle size obtained was 253 nm.

### Example 6

The purpose of this example was to prepare a nanoparticulate anhydrous docetaxel formulation.

An aqueous dispersion of 1% (w/w) anhydrous docetaxel was combined with 0.25% (w/w) Pluronic® F127. This mixture was then milled in a 15 mL glass bottle using a low energy roller mill (U.S. Stoneware, Mahwah, N.J.) along with 0.5 mm ceramic media (Tosoh, Ceramics Division) (50% media load). The mixture was milled at a speed of 130 rpms for 72 hours.

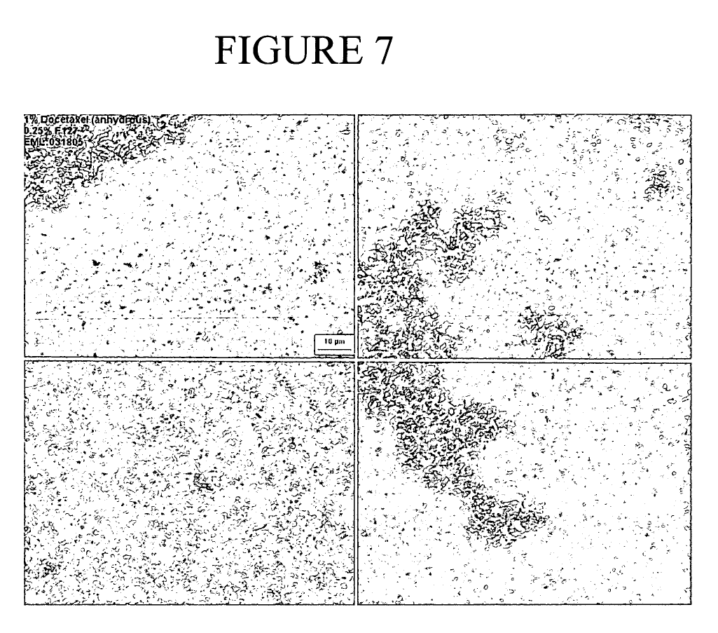

The particle size of the milled docetaxel particles was measured, in deionized distilled water, using a Horiba LA 910 particle size analyzer. The mean milled docetaxel particle size was 56.42 microns, with a D50 of 65.55 microns, and a D90 of 118.5 microns. Because of the large particle size of the milled sample, the sample was then sonicated for 30 seconds to determine if aggregated docetaxel particles were present. Following 30 seconds of sonication, the mean milled docetaxel particle size was 1.468 microns, with a D50 of 330 nm and a D90 of 5.18 microns. FIG. 7 shows a light micrograph of the milled doectaxel.

The results demonstrate that at the particular concentrations of drug and surface stabilizer utilized, Pluronic® F127 does not successfully stabilize anhydrous docetaxel.

### Example 7

The purpose of this example was to prepare a nanoparticulate trihydrate docetaxel formulation.

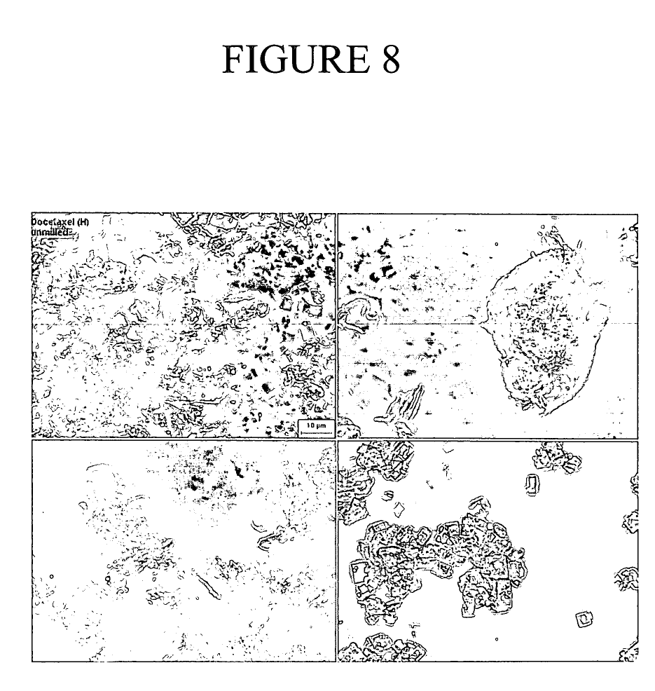

FIG. 8 shows a light micrograph of unmilled trihydrate docetaxel. Unmilled trihydrate docetaxel has a mean particle size of 61,610 nm, with a D50 of 51,060 nm and a D90 of 119,690 nm.

An aqueous dispersion of 5% (w/w) trihydrate docetaxel (Camida Ltd.) was combined with 1.25% (w/w) polyvinylpyrrolidone (PVP) K12 and 0.25% (w/w) sodium deoxycholate. The mixture was then milled in a 10 ml chamber of a NanoMill® 0.01 (NanoMill Systems, King of Prussia, Pa.; see e.g., U.S. Pat. No. 6,431,478), along with 220 micron PolyMill® attrition media (Dow Chemical) (89% media load). The mixture was milled at a speed of 2500 rpms for 60 minutes.

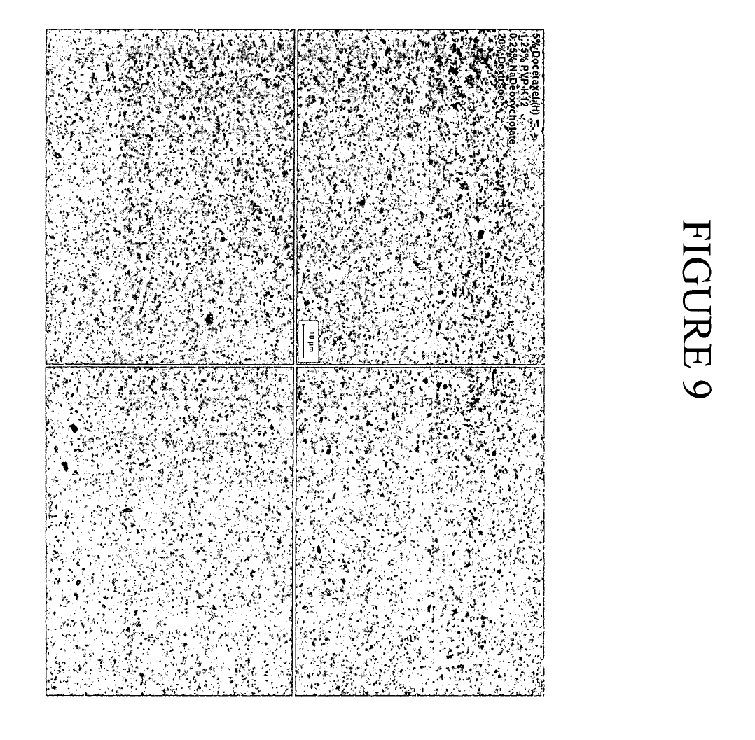

Following milling, the particle size of the milled docetaxel particles was measured, in deionized distilled water, using a Horiba LA 910 particle size analyzer. The mean milled docetaxel particle size was 152 nm, with a D50 of 141 nm and a D90 of 202 nm. FIG. 9 shows a light micrograph of the milled doectaxel.

The results demonstrate the successful preparation of a stable nanoparticulate docetaxel formulation, as the mean particle size obtained was 152 nm.

### Example 8

The purpose of this example was to prepare a nanoparticulate trihydrate docetaxel formulation.

An aqueous dispersion of 5% (w/w) trihydrate docetaxel was combined with 1.25% (w/w) polyvinylpyrrolidone (PVP) K17, 0.25% (w/w) sodium deoxycholate, and 20% (w/w) dextrose. This mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2900 rpms for 60 minutes.

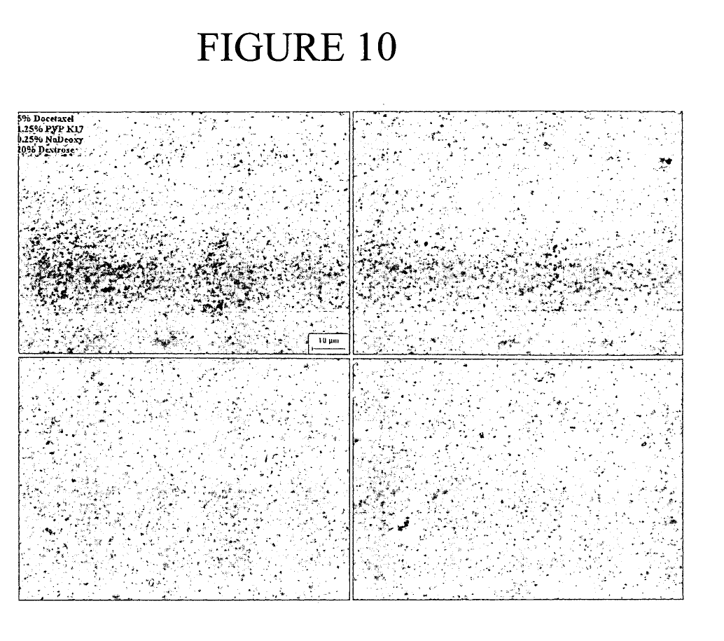

Following milling, the particle size of the milled docetaxel particles was measured, in deionized distilled water, using a Horiba LA 910 particle size analyzer. The mean milled docetaxel particle size was 113 nm, with a D50 of 109 nm and a D90 of 164 nm. FIG. 10 shows a light micrograph of the milled doectaxel.

The results demonstrate the successful preparation of a stable nanoparticulate docetaxel formulation, as the mean particle size obtained was 164 nm.

### Example 9

The purpose of this example was to determine the long term stability of the nanoparticulate trihydrate docetaxel formulation prepared in Example 8.

The nanoparticulate trihydrate docetaxel formulation prepared in Example 8, comprising 5% (w/w) trihydrate docetaxel, 1.25% (w/w) polyvinylpyrrolidone (PVP) K17, 0.25% (w/w) sodium deoxycholate, and 20% (w/w) dextrose, was stored in the cold (<15° C.) for 6 months.

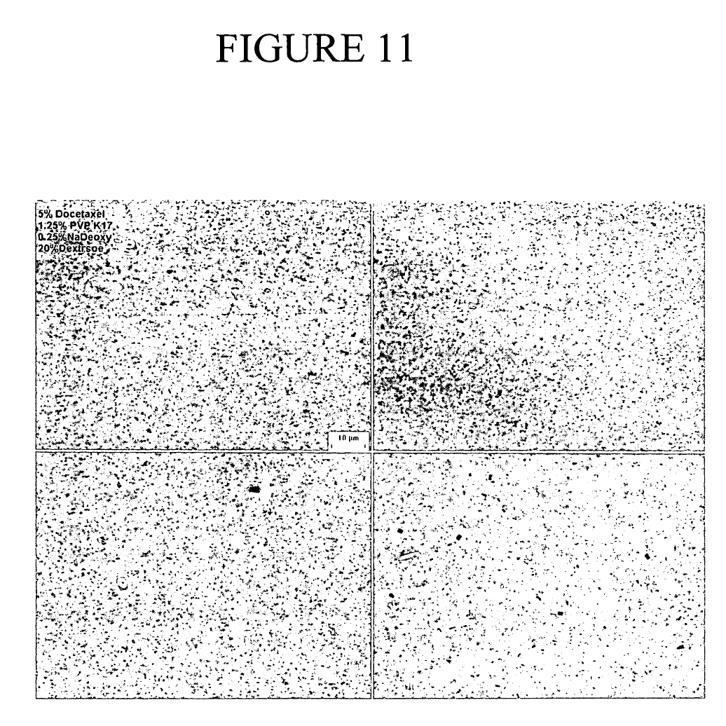

Following the six month storage period, the particle size of the docetaxel particles was measured, in deionized distilled water, using a Horiba LA 910 particle size analyzer. The mean docetaxel particle size was 147 nm, with a D50 of 136 nm and a D90 of 205 nm. FIG. 11 shows a light micrograph of the doectaxel composition following cold storage for 6 months.

The results indicate that the nanoparticulate docetaxel compositions can be stored for extensive periods of time without significant particle size growth.

### Example 10

The purpose of this example was to prepare a nanoparticulate trihydrate docetaxel formulation.

An aqueous dispersion of 5% (w/w) trihydrate docetaxel was combined with 1.25% (w/w) Tween® 80, 0.1 % (w/w) lecithin, and 20% (w/w) dextrose. This mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2900 rpms for 75 minutes.

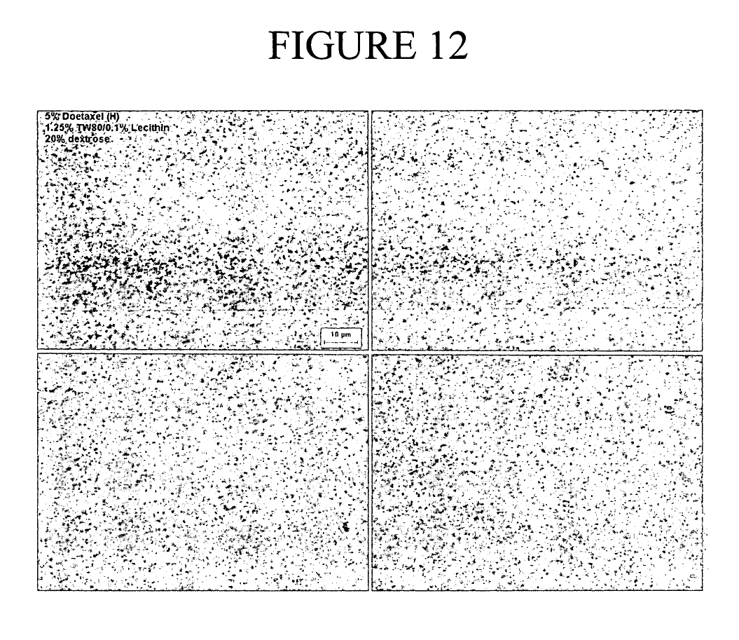

Following milling, the particle size of the milled docetaxel particles was measured, in deionized distilled water, using a Horiba LA 910 particle size analyzer. The mean milled docetaxel particle size was 144 nm, with a D50 of 137 nm and a D90 of 193 nm. FIG. 12 shows a light micrograph of the milled doectaxel.

The results demonstrate the successful preparation of a stable nanoparticulate docetaxel formulation, as the mean particle size obtained was 144 nm.

### Example 11

The purpose of this example was to test the long term stability of the nanoparticulate trihydrate docetaxel formulation prepared in Example 10.

The nanoparticulate trihydrate docetaxel formulation prepared in Example 10, comprising 5% (w/w) trihydrate docetaxel, 1.25% (w/w) Tween® 80, 0.1% lecithin (w/w), and 20% (w/w) dextrose, was stored in the cold (<15° C.) for 6 months.

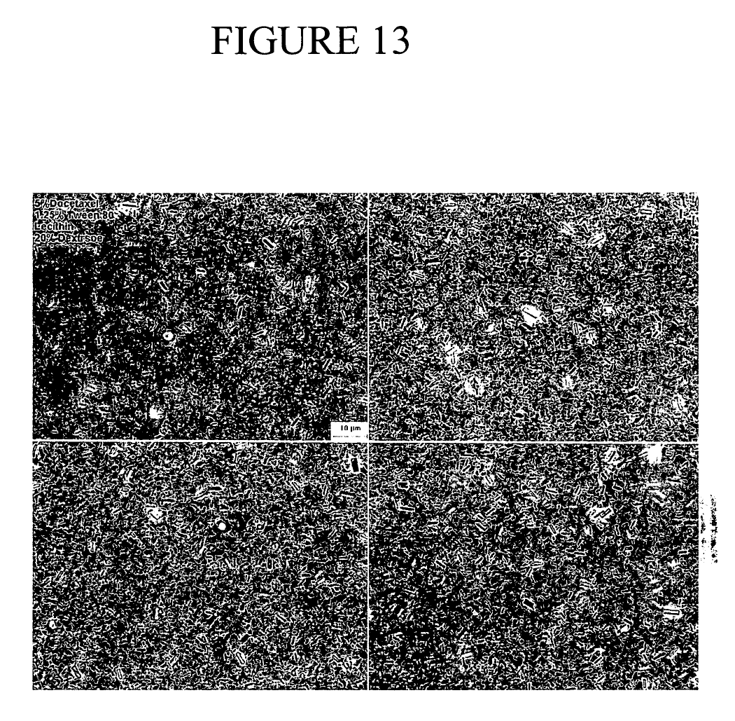

Following the six month storage period, the particle size of the docetaxel particles was measured, in deionized distilled water, using a Horiba LA 910 particle size analyzer. The mean docetaxel particle size was 721 nm, with a D50 of 371 nm and a D90 of 1.76 microns. FIG. 13 shows a light micrograph of the doectaxel composition following cold storage for 6 months.

The results indicate that the nanoparticulate docetaxel compositions can be stored for extensive periods of time while still maintaining an effective average particle size of less than 2 microns.

### Example 12

The purpose of this example was to prepare a nanoparticulate trihydrate docetaxel formulation.

An aqueous dispersion of 5% (w/w) trihydrate docetaxel was combined with 1.25% (w/w) TPGS (Vitamin E PEG) and 0.1 % (w/w) sodium deoxycholate. This mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 120 minutes.

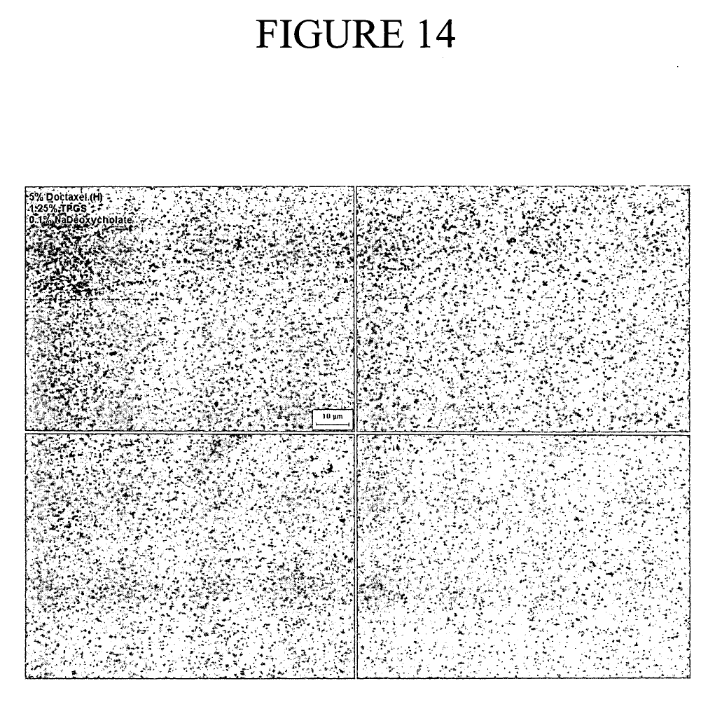

Following milling, the particle size of the milled docetaxel particles was measured, in deionized distilled water, using a Horiba LA 910 particle size analyzer. The mean milled docetaxel particle size was 134 nm, with a D50 of 129 nm and a D90 of 179 nm. FIG. 14 shows a light micrograph of the milled doectaxel.

The results demonstrate the successful preparation of a stable nanoparticulate docetaxel formulation, as the mean particle size obtained was 134 nm.

### Example 13

The purpose of this example was to prepare a nanoparticulate trihydrate docetaxel formulation.

An aqueous dispersion of 5% (w/w) trihydrate docetaxel was combined with 1.25% (w/w) Pluronic® F 108, 0.1% (w/w) sodium deoxycholate, and 10% (w/w) dextrose (w/w). The mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 120 minutes.

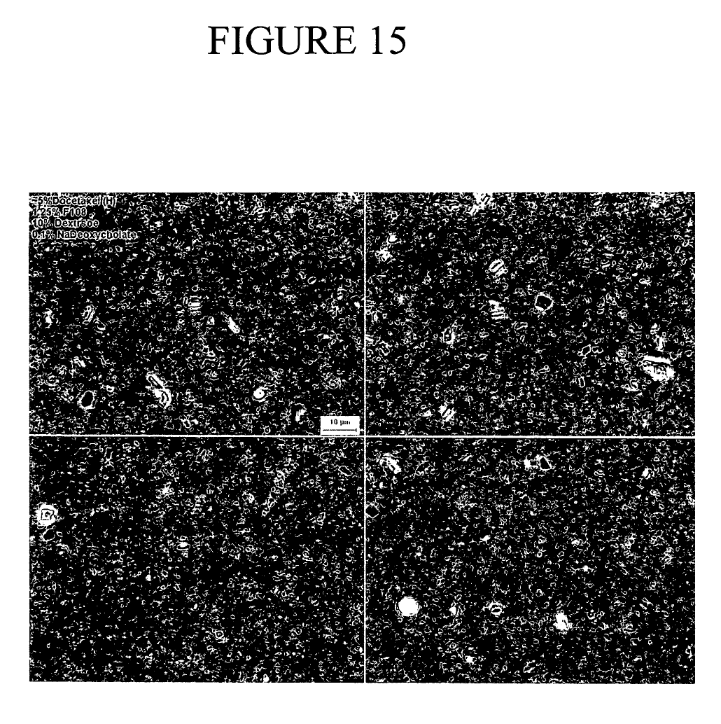

Following milling, the particle size of the milled docetaxel particles was measured, in deionized distilled water, using a Horiba LA 910 particle size analyzer. The mean milled docetaxel particle size was 632 nm, with a D50 of 172 nm and a D90 of 601 nm. FIG. 15 shows a light micrograph of the milled doectaxel.

The results demonstrate the successful preparation of a stable nanoparticulate docetaxel formulation, as the mean particle size obtained was 632 nm.

### Example 14

The purpose of this example was to prepare a nanoparticulate docetaxel formulation.

An aqueous dispersion of 5% (w/w) docetaxel was combined with 1.25% (w/w) Plasdone® S630 and 0.05% (w/w) dioctylsulfosuccinate (DOSS). The mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 60 minutes.

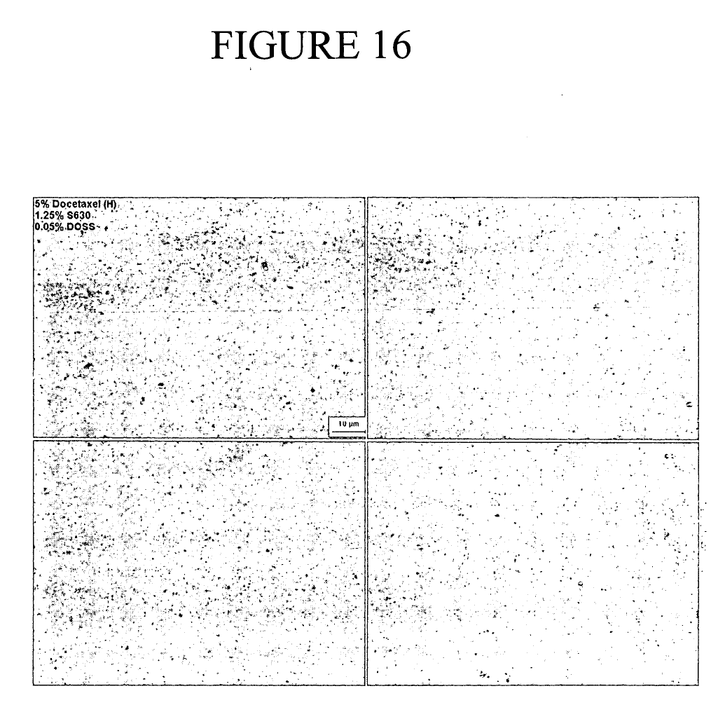

Following milling, the particle size of the milled docetaxel particles was measured, in deionized distilled water, using a Horiba LA 910 particle size analyzer. The mean milled docetaxel particle size was 142 nm, with a D50 of 97.8 nm and a D90 of 142 nm. FIG. 16 shows a light micrograph of the milled doectaxel.

The results demonstrate the successful preparation of a stable nanoparticulate docetaxel formulation, as the mean particle size obtained was 142 nm.

### Example 15

The purpose of this example was to prepare a nanoparticulate docetaxel formulation.

An aqueous dispersion of 5% (w/w) docetaxel was combined with 1.25% (w/w) HPMC and 0.05% (w/w) dioctylsulfosuccinate (DOSS). The mixture was then milled in a 10 ml chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 60 minutes.

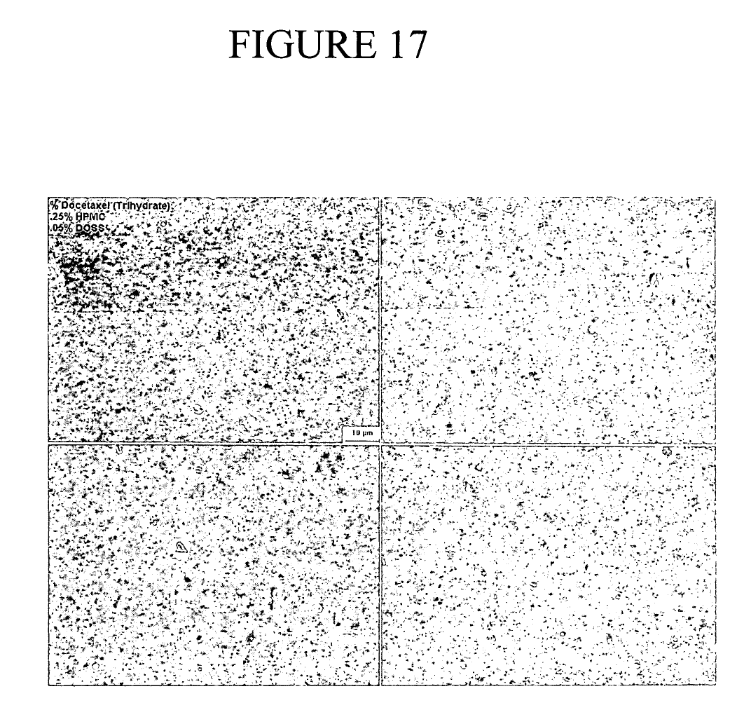

Following milling, the particle size of the milled docetaxel particles was measured, in deionized distilled water, using a Horiba LA 910 particle size analyzer. The mean milled docetaxel particle size was 157 nm, with a D50 of 142 nm and a D90 of 207 nm. FIG. 17 shows a light micrograph of the milled doectaxel.

The results demonstrate the successful preparation of a stable nanoparticulate docetaxel formulation, as the mean particle size obtained was 157 nm.

### Example 16

The purpose of this experiment was to determine the maximum tolerated dose of a nanoparticulate docetaxel formulation.

To evaluate and characterize the acute toxicity of nanoparticulate docetaxel formulations, two nanoparticulate dispersions were utilized. (1) a nanoparticulate dispersion of docetaxel having PVP and sodium deoxycholate as surface stabilizers (prepared in Example 8); and (2) a nanoparticulate dispersion of docetaxel having Tween® 80 and lecithin as surface stabilizers (prepared in Example 10).

Both nanoparticulate docetaxel formulations were administered intravenously at various doses to mice. The maximum tolerated dose (MD) for both nanoparticulate docetaxel formulations was 500 mg/kg.

The commercially available non-nanoparticulate docetaxel product, TAXOTERE®, was also tested in parallel with the nanoparticulate docetaxel formulations. The MD for TAXOTERE® was 40 mg/kg.

Thus, nanoparticulate formulations of docetaxel are well tolerated and can be administered at significantly higher doses than conventional, non-nanoparticulate docetaxel formulations.

### Example 17

The purpose of this example was to prepare a nanoparticulate docetaxel formulation.

An aqueous dispersion of 5% (w/w) anhydrous docetaxel was combined with 1% (w/w) albumin and 0.5% (w/w) sodium deoxycholate. The mixture was then milled in a 10 mL chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 5.5 hours.

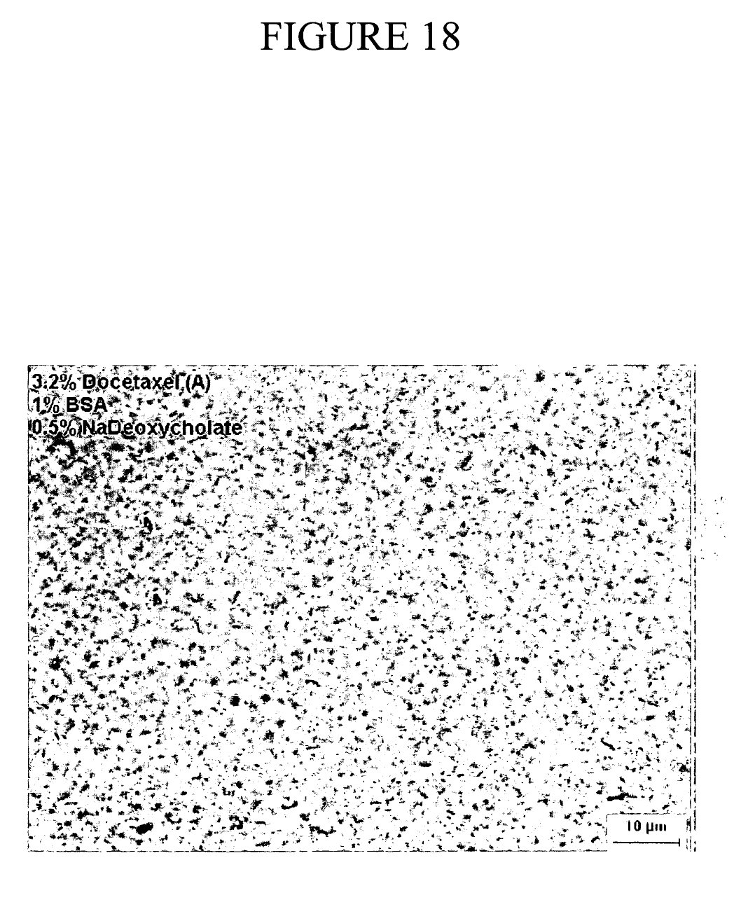

Following milling, the particle size of the milled docetaxel particles was measured, in deionized distilled water, using a Horiba LA 910 particle size analyzer. The mean milled docetaxel particle size was 271 nm, with a D90 of 480 nm. FIG. 18 shows a light micrograph of the milled doectaxel.

The results demonstrate the successful preparation of a stable nanoparticulate docetaxel formulation, as the mean particle size obtained was 271 nm.

### Example 18

The purpose of this example was to prepare a nanoparticulate docetaxel formulation.

An aqueous dispersion of 5% (w/w) trihydrate docetaxel was combined with 1% (w/w) albumin and 0.5% (w/w) sodium deoxycholate. The mixture was then milled in a 10 mL chamber of a NanoMill® 0.01 (NanoMill Systems, King of Prussia, Pa.), along with 220 micron PolyMill® attrition media (Dow Chemical) (89% media load). The mixture was milled at a speed of 2500 rpms for 60 min.

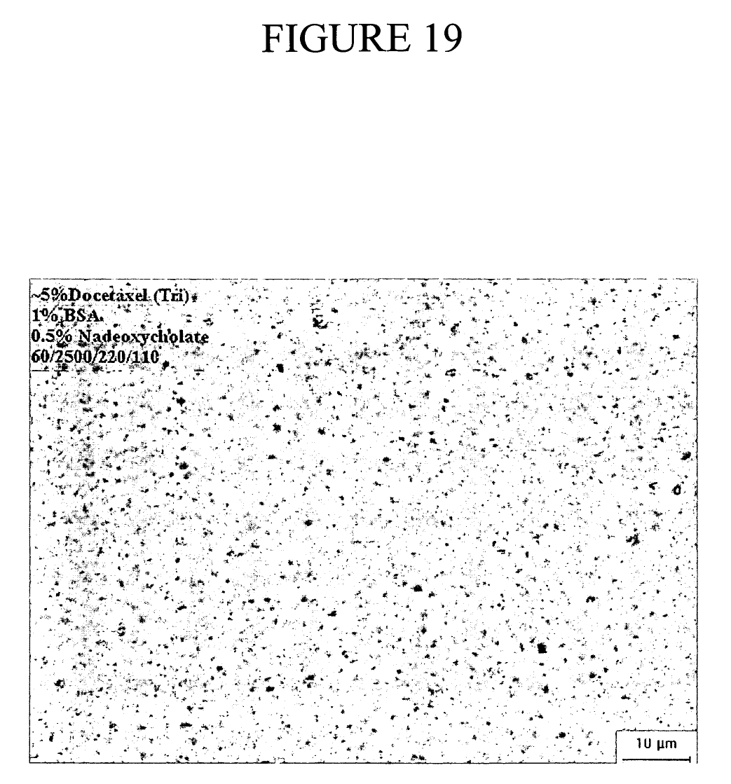

Following milling, the particle size of the milled docetaxel particles was measured, in deionized distilled water, using a Horiba LA 910 particle size analyzer. The mean milled docetaxel particle size was 174 nm, with a D90 of 252 nm. FIG. 19 shows a light micrograph of the milled doectaxel.

The results demonstrate the successful preparation of a stable nanoparticulate docetaxel formulation, as the mean particle size obtained was 174 nm.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A composition comprising:
(a) particles of docetaxel or an analogue thereof having an effective average particle size of less than about 2000 nm; and
(b) at least one surface stabilizer.

2. The composition of claim 1, wherein the docetaxel or analogue thereof is selected from the group consisting of a crystalline phase, an amorphous phase, a semi-crystalline phase, a semi-amorphous phase, and mixtures thereof.

3. The composition of claim 1, wherein the effective average particle size of the particles of the docataxel or analogue thereof is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

4. The composition of claim 1, wherein the composition is formulated:
(a) for administration selected from the group consisting of oral, pulmonary, rectal, opthalmic, colonic, parenteral, intracisternal, intravaginal, intraperitoneal, local, buccal, nasal, and topical administration;
(b) into a dosage form selected from the group consisting of liquid dispersions, solid dispersions, liquid-filled capsule, gels, aerosols, ointments, creams, lyophilized formulations, tablets, capsules, multi-particulate filled capsule, tablet composed of multi-particulates, compressed tablet, and a capsule filled with enteric-coated beads of a docetaxel or analogue thereof,
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c).

5. The composition of claim 4, wherein the composition is an injectable formulation

6. The composition of claim 1, wherein:
(a) the surface stabilizer is present in an amount selected from the group consisting of about 0.5% to about 99.999%, about 5.0% to about 99.9%, and about 10% to about 99.5%, by weight, based on the total combined dry weight of the docetaxel or analogue thereof and at least one surface stabilizer, not including other excipients;
(b) the docetaxel or analogue thereof is present in an amount selected from the group consisting of about 99.5% to about 0.001%, about 95% to about 0.1%, and about 90% to about 0.5%, by weight, based on the total combined weight of the docetaxel or analogue thereof and at least one surface stabilizer, not including other excipients; or
(c) a combination of (a) and (b).

7. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

8. The composition of claim 1, wherein the at least one surface stabilizer is selected from the group consisting of cetyl pyridinium chloride, albumin, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

9. The composition of claim 1, additionally comprising one or more non-docetaxel or analogue thereof active agents.

10. The composition of claim 1, wherein upon administration to a mammal the docetaxel or analogue thereof particles redisperse such that the particles have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

11. The composition of claim 1, wherein the composition redisperses in a biorelevant media such that the docetaxel or analogue thereof particles have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

12. The composition of claim 11, wherein the biorelevant media is selected from the group consisting of water, aqueous electrolyte solutions, aqueous solutions of a salt, aqueous solutions of an acid, aqueous solutions of a base, and combinations thereof.

13. The composition of claim 1, wherein the Tₘₐₓ of the docetaxel or analogue thereof, when assayed in the plasma of a mammalian subject following administration, is less than the Tₘₐₓ for a non-nanoparticulate docetaxel or analogue thereof formulation, administered at the same dosage.

14. The composition of claim 13, wherein the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate docetaxel or analogue thereof formulation, administered at the same dosage.

15. The composition of claim 13, wherein the composition exhibits a Tₘₐₓ selected from the group consisting of less than about 6 hours, less than about 5 hours, less than about 4 hours, less than about 3 hours, less than about 2 hours, less than about 1 hour, and less than about 30 minutes after administration to fasting subjects.

16. The composition of claim 1, wherein the Cₘₐₓ of the docetaxel or analogue thereof, when assayed in the plasma of a mammalian subject following administration, is greater than the Cₘₐₓ for a non-nanoparticulate docetaxel or analogue thereof formulation, administered at the same dosage.

17. The composition of claim 16, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate formulation of docetaxel or analogue thereof, administered at the same dosage.

18. The composition of claim 1, wherein the AUC of the docetaxel or analogue thereof, when assayed in the plasma of a mammalian subject following administration, is greater than the AUC for a non-nanoparticulate docetaxel or analogue thereof formulation, administered at the same dosage.

19. The composition of claim 18, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of docetaxel or analogue thereof, administered at the same dosage.

20. The composition of claim 1 which does not produce significantly different absorption levels when administered under fed as compared to fasting conditions.

21. The composition of claim 20, wherein the difference in absorption of the docetaxel or analogue thereof composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

22. The composition of claim 1, wherein administration of the composition to a human in a fasted state is bioequivalent to administration of the composition to a subject in a fed state.

23. The composition of claim 22, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

24. The composition of claim 1, wherein the docetaxel analogue is selected from the group consisting of:
(a) docetaxel analogues comprising cyclohexyl groups instead of phenyl groups at the C-3′ benzoate position, the C-2 benzoate positions, or a combination thereof;
(b) docetaxel analogues lacking phenyl or an aromatic group at C-3′ or C-2 position;
(c) 2-amido docetaxel analogues;
(d) docetaxel analogues lacking the oxetane D-ring but possessing the 4alpha-acetoxy group;
(e) 5(20)deoxydocetaxel;
(f) 10-deoxy-10-C-morpholinoethyl docetaxel analogues;
(g) analogues having a t-butyl carbamate as the isoserine N-acyl substituent, but differing from docetaxel at C-10 (acetyl group versus hydroxyl) and at the C-13 isoserine linkage (enol ester versus ester);
(h) docetaxel analogues having a peptide side chain at C3;
(i) XRP9881 (10-deacetyl baccatin III docetaxel analogue);
(j) XRP6528 (10-deacetyl baccatin III docetaxel analogue);
(k) Ortataxel (14-beta-hydroxy-deacetyl baccatin III docetaxel analogue);
(l) MAC-321 (10-deacetyl-7-propanoyl baccatin docetaxel analogue);
(m) DJ-927 (7-deoxy-9-beta-dihydro-9,10, O-acetal taxane docetaxal analogue);
(n) docetaxel analogues having C2-C3′N-linkages bearing an aromatic ring at position C2, and tethered between N3′ and the C2-aromatic ring at the ortho position;
(o) docetaxel analogues having C2-C3′N-linkages bearing an aromatic ring at position C2, and tethered between N3′ and the C2-aromatic ring at the meta position;
(p) docetaxel analogues bearing 22-membered (or more) rings connecting the C-2 OH and C-3′ NH moieties;
(q) 7beta-O-glycosylated docetaxel analogues;
(r) 10-alkylated docetaxel analogues;
(s) 2′,2′-difluoro docetaxel analogues;
(t) 3′-(2-furyl) docetaxel analogues;
(u) 3′-(2-pyrrolyl) docetaxel analogues; and
(v) fluorescent and biotinylated docetaxel analogues.

25. The composition of claim 24, wherein the docetaxel analogue is selected from the group consisting of:
(a) 3′-dephenyl-3′cyclohexyldocetaxel;
(b) 2-(hexahydro)docetaxel;
(c) 3′-dephenyl-3′cyclohexyl-2-(hexahydro)docetaxel;
(d) 3′-dephenyl-3′-cyclohexyldocetaxel;
(e) 2-(hexahydro)docetaxel;
(f) m-methoxy docetaxel analogues;
(g) m-chlorobenzoylamido docetaxel analogues;
(h) 5(20)-thia docetaxel analogues;
(i) doctaxel analogues in which the 7-hydroxyl group is modified to the hydrophobic group methoxy;
(j) doctaxel analogues in which the 7-hydroxyl group is modified to the hydrophobic group deoxy;
(k) doctaxel analogues in which the 7-hydroxyl group is modified to the hydrophobic group 6,7-olefin;
(1) doctaxel analogues in which the 7-hydroxyl group is modified to the hydrophobic group alpha-F;
(m) doctaxel analogues in which the 7-hydroxyl group is modified to the hydrophobic group 7-beta-8-beta-methano;
(n) doctaxel analogues in which the 7-hydroxyl group is modified to the hydrophobic group fluoromethoxy;
(o) 10-alkylated docetaxel analogue having a methoxycarbonyl group at the end of the alkyl moiety;
(p) docetaxel analogues that possess a N-(7-nitrobenz-2-oxa-1,3-diazo-4-yl)amido-6-caproyl chain in position 7 or 3′;
(q) docetaxel analogues that possess a N-(7-nitrobenz-2-oxa-1,3-diazo-4-yl)amido-3-propanoyl group at 3′; and
(r) docetaxel analogues that possess a 5′-biotinyl amido-6-caproyl chain in position 7, 10 or 3′.

26. A method of treating a cancer comprising administering to a mammal an effective amount of a composition comprising:
(a) particles of a docetaxel or analogue thereof having an effective average particle size of less than about 2000 nm; and
(b) at least one surface stabilizer.

27. The method of claim 26, wherein the composition is formulated for administration by injection.

28. The method of claim 26, wherein the cancer is selected from the group consisting of breast, prostate, ovarian, and lung.

29. A method of making a nanoparticulate docetaxel or analogue thereof composition comprising contacting particles of docetaxel or an analogue thereof with at least one surface stabilizer for a time and under conditions sufficient to provide a docetaxel or analogue thereof composition having an effective average particle size of less than about 2000 nm.

30. The method of claim 29, wherein the contacting comprises grinding, homogenizing, precipitation, or supercritical fluids processing.
